(19) United States
(12) Patent Application Publication
(10) Pub. No.: US 2004/0115195 A1
(43) Pub. Date: Jun. 17, 2004

(54) Pharmaceutical compositions and methods of using secreted frizzled related protein

(76) Inventor: Peter Bodine, Havertown, PA (US)

Correspondence Address: DARBY & DARBY P.C., P.O. BOX 5257, NEW YORK, NY, 10150-5257, US

(21) Appl. No.: 10/666,851

(22) Filed: Sep. 19, 2003

Related U.S. Application Data
(63) Continuation-in-part of application No. 10/169,545, filed on May 31, 2002.
(60) Provisional application No. 60/412,379, filed on Sep. 19, 2002.

(51) Int. Cl. A61K 39/395; A61K 38/17; A61K 48/00

(52) U.S. Cl. 424/144.1; 514/12; 514/44

(57) ABSTRACT

Pharmaceutical compositions and methods of use in regulation of mammalian bone forming activities of sFRPs (secreted frizzled-related proteins) are disclosed. sFRPs are secreted receptors for Wnts, which are important polypeptide growth factors that are known to regulate fundamental biological processes like tissue polarity, embryonic development, and tumorigenesis. A sFRP was isolated from human osteoblast cells and identified as sFRP-1 (also known as SARP-2) and shown to be regulated by osteogenic agents in hOB cells in a differentiation selective manner, modulating the life of osteoblasts/preosteocytes. An sFRP-1 knock-out mouse was generated and deletion of sFRP-1 was found to not affect nonskeletal tissues, skeletal morphology or cortical bone development, while resulting in increased trabecular bone formation, decreased osteoblast and osteocyte apoptosis and increased osteoprogenitor differentiation.

This application is a continuation-in-part of U.S. patent application Ser. No. 10/169,545, filed May 31, 2002. This application also claims priority from U.S. Provisional Patent Application Serial No. 60/412,379, filed Sep. 19, 2002. Each of these applications is incorporated herein by reference in its entirety.

### FIELD OF THE INVENTION

The present invention relates to compositions and methods for regulating bone-forming activity. More particularly, the present invention relates to methods and pharmaceutical compositions for regulating bone forming activity with a secreted frizzled related protein (sFRP) derived from an osteoblast cell line, portions thereof, as well as antibodies and nucleic acids, including antisense, based thereon.

### BACKGROUND OF THE INVENTION

The topic of bone formation regulation and bone-related disorders has recently gained considerable attention. For example, in the women's health area there has been a particular focus on the bone-related disorder osteoporosis. Throughout life, there is a constant remodeling of skeletal bone. Bone is formed and maintained by two cell types: osteoblasts that synthesize and mineralize the bone matrix, and osteoclasts that resorb the calcified tissue (Komm and Bodine, Osteoporosis 2001, R. Marcus, F. D., and J. Kelsey, eds. (San Diego, Academic Press), 305-337; Bodine and Komm, Vita. Horm. 2002, 64:101-151; Goltzman, Nat. Rev. Drug Disc. 2002, 1:784-796). Osteoblasts arise from multipotent mesenchymal stem cells that are located in bone marrow (Lian et al., Primer on the metabolic bone diseases and disorders of mineral metabolism 1999, M. J. Favus, ed. (Philadelphia, Lippincott Williams & Wilkins), 14-29; Bodine and Komm, Vita. Horm. 2002, 64:101-151; Goltzman, Nat. Rev. Drug Disc. 2002, 1:784-796), while osteoclasts originate from hematopoietic bone marrow cells (Teitelbaum, Science 2000, 289:1504-1508; Goltzman, Nat. Rev. Drug Disc. 2002 1:784-796). These cells work together in a process known as bone remodeling, which is the mechanism by which immature, damaged or aged bone is replaced with new lamellar bone (Mundy, Primer on the metabolic bone diseases and disorders of mineral metabolism 1999, M. J. Favus, ed. (Philadelphia, Lippincott Williams & Wilkins), 199:30-38). Bone remodeling is initiated by recruitment and activation of osteoclasts that remove the mineralized matrix. The process ends about 6 months later with the filling-in of the resorption pit with newly formed osteoid by the osteoblasts. At the end of this last phase, the bone-forming cells experience one of three fates (Manolagas, Endocr. Rev. 2000, 21:115-137; Bodine and Komm, Vita. Horm. 2002, 64:101-151; Goltzman, Nat. Rev. Drug Disc. 2002, 1;784-796). They can differentiate to osteocytes upon entrapment within the mineralized matrix; they can differentiate to quiescent lining cells; or they can undergo apoptosis (Manolagas, Endocr. Rev. 2000, 21:115-137).

The majority of studies on age-related changes in human bone have been directed towards elucidating changes in bone on a morphological level or by quantitatively comparing rates of bone loss. Disruption of the fine balance between the differentiation of new osteoclast and osteoblast cells, and the timing of cell death by apoptosis is thought to be an important mechanism behind bone loss disorders such as osteogenesis. Therapeutic agents that alter the prevalence of apoptosis in osteoblasts and/or osteoclasts are useful and desirable to correct the imbalance in cell numbers that is the basis of diminished bone mass and increased risk of fractures in osteoporosis (for review, see, Manolagas, Endocrine Reviews 2002, 21:115-137; and Weinstein and Manolagas, Am. J. Med. 2000, 108:153-164).

The identification of the mechanisms involved in bone disorders is crucial for the understanding of bone physiology and bone disorders. While numerous genes and gene families and the polypeptides encoded by them that participate in the regulation of bone cells have been identified and cloned, their functions have not been clearly delineated due to the complexities of the bone formation pathways.

### The Wnt Gene Family

One group of genes and the proteins encoded by them that play an important role in regulating cellular development is the Wnt family of glycoproteins. Wnt proteins are a family of growth factors consisting of more than a dozen structurally related molecules that are involved in the regulation of fundamental biological processes like apoptosis, embryogenesis, organogenesis, morphogenesis and tumorigenesis (reviewed in Nusse and Varmus, Cell 1992, 69:1073-1087). These polypeptides are multipotent factors and have similar biological activities to other secretory proteins like transforming growth factor (TGF)-β, fibroblast growth factors (FGFs), nerve growth factor (NGF), and bone morphogenetic proteins (BMPs). One member of the Wnt growth factor family, termed Wnt-x, is preferentially expressed in bone tissue and in bone-derived cells and appears to be involved in maintaining the mature osteoblast (bone-forming cell) phenotype (WO 95/17416).

### The Frizzled Family of Proteins

Studies indicate that certain Wnt proteins interact with a family of proteins named “Frizzled” that act as receptors for Wnt proteins or as components of a Wnt receptor complex (reviewed in Moon et al., Cell 1997, 88:725-728; Barth et al., Curr. Opin. Cell Biol. 1997, 9:683-690). Frizzled proteins contain an amino terminal signal sequence for secretion, a cysteine-rich domain (CRD) that is thought to bind Wnt, seven putative transmembrane domains that resemble a G-protein coupled receptor, and a cytoplasmic carboxyl terminus.

The discovery of the first secreted frizzled-related protein (sFRP) was reported by Hoang et al. (J. Biol. Chem. 1996, 271:26131-26137). This protein, which was called “Frzb” for frizzled motif in bone development, was purified and cloned from bovine articular cartilage extracts based on its ability to stimulate in vivo chondrogenic activity in rats. The human homologue of the bovine gene was also cloned. However, unlike the frizzled proteins, Frzb did not contain a serpentine transmembrane domain. Thus, this new member of the frizzled family appeared to be a secreted receptor for Wnt. The Frzb cDNA encoded for a 325 amino acid/36,000 Dalton protein and was predominantly expressed in the appendicular skeleton. The highest level of expression was in developing long bones and corresponded to epiphyseal chondroblasts; expression then declined and disappeared toward the ossification center.

Recent studies indicate sFRPs participate in apoptosis. Thus, some sFRPs have been identified as “SARPs” for secreted apoptosis related proteins. Additional members of the sFRP family have also recently been identified and shown to be antagonists of Wnt action. There are currently at least five known human sFRP/SARP genes: sFRP-1/FrzA/FRP-1/SARP-2, sFRP-2/SDF-5/SARP-1, sFRP-3/Frzb-1/FrzB/Fritz, sFRP-4 and sFRP-5/SARP-3 (Leimeister et al., Mechanisms of Development 1998, 75:29-42, which sequences of this reference are incorporated herein). Although the precise role that SARPs/sFRPs play in apoptosis is not yet clear, these proteins appear to either suppress or enhance the programmed cell death process.

In summary, a need exists for the definitive identification of targets for the treatment of bone disorders, including bone resorption disorders such as osteoporosis and Paget's disease, and for regulation of bone formation in humans.

### SUMMARY OF THE INVENTION

The present invention provides methods and pharmaceutical compositions for regulating bone-forming activity in a mammal. These methods and compositions involve use of a secreted frizzled related protein (sFRP) or a regulating portion thereof. Additional compositions of the present invention employ antibodies against such proteins or portions thereof, and alternatively can employ nucleic acids that encode such proteins or portions thereof or that are complementary to the coding sequence of such a nucleic acid sequence. Such nucleic acids include antisense sequences, siRNAs and shRNAs that block or inhibit sFRP expression. Further additional compounds include ribozymes that can inhibit expression of sFRPs. The bone forming activities regulated by the composition of the present invention include the regulation of bone growth and bone density.

In a specific embodiment, the sFRP is from human osteoblast cells. In a further embodiment, the sFRP is sFRP-1. sFRP-1 can have the amino acid sequence set forth in SEQ ID NO: 2, e.g., as obtained by the expression of the polynucleotide sequence set forth in SEQ ID NO: 1 or can have an amino acid sequence that is about at least 90% identical to SEQ ID NO: 2, as obtained by the expression of a polynucleotide sequence that is about at least 85% identical to SEQ ID NO: 1, and allelic variants and muteins of these polynucleotides and polypeptides.

In another aspect, methods of the present invention include methods for treating a bone disorder in a mammal, particularly a human. These methods comprise administering a pharmaceutical composition as described above. The methods for treating a bone disorder include but are not limited to disorders comprising the group consisting of (a) a bone formation disorder, (b) a bone resorption disorder, and (c) a bone density disorder.

In another aspect of the invention, the bone disorder is a degenerative bone disorder. Examples of degenerative bone disorders include osteodegeneration disorders. The osteodegeneration disorder includes, but is not limited to, osteopenia, osteoarthritis, and osteoporosis.

The invention also includes methods for preventing a bone disorder in a mammal, particularly in a human who may be a healthy individual (i.e., not currently suffering from or having a bone disorder) but who is susceptible to or is at risk of developing a bone disorder in the future. Examples of such individuals include a young adult, an adolescent, a juvenile, or an infant. The methods for preventing a bone disorder can be used to prevent, but are not limited to, any of the bone disorders described herein. In specific embodiments the bone disorder prevented by these methods is a degenerative bone disorder, particularly osteoporosis such as Type II or “senile” osteoporosis.

In specific embodiments the methods for treating and/or preventing bone disorders inhibit the expression and/or activity of a sFRP in the mammal, for example sFRP-1. Thus, the pharmaceutical compositions of the invention are present in a therapeutically effective amount. For example, the pharmaceutical compositions and methods of the invention inhibit sFRP expression and/or activity by at least 10%, more preferably by at least 20% and most preferably by at least 30%, 40%, 50%, 60%, 70%, 80%, 90%, or 95%. In particularly preferred embodiments, the methods and pharmaceutical compositions of the invention will completely or almost completely inhibit (i.e., abolish) the sFRP activity and/or expression.

In an additional embodiment, the invention includes methods for identifying test compounds capable of regulating sFRP activity. In the method, the compounds that regulate the bone-forming activity in a mammal are assayed by first incubating a sample comprising a sFRP in a medium containing the test compound. The next step is to determine the sFRP activity, wherein an increase in activity relative to sFRP alone indicates the compound is a sFRP activator and a decrease in activity indicates the compound is a sFRP inhibitor.

In a further embodiment, the invention includes methods of modulating Wnt-mediated signaling in a cell. This method involves contacting the cell with the sFRP described above, whereby Wnt activity is regulated.

The present invention additionally relates to a method of facilitating bone formation or repair in bone cells. In this embodiment, the method involves isolating the cells from a bone culture, introducing a recombinant construct expressing sFRP-1 into bone cells. The cells can be in bone tissue in vivo, or they can be isolated, either from bone tissue or from a bone culture, and implanted in the bone tissue (ex vivo). Preferably, the construct expresses an antisense sequence for a nucleic acid sequence that encodes all or a portion of a sFRP protein or contains an siRNA, shRNA or ribozyme sequence that inhibits sFRP expression. Alternatively, the construct encodes for an antibody that neutralizes sFRP activity. In a further embodiment, the construct encodes for sFRP, resulting in an increased amount of sFRP expression and activity.

Another embodiment relates to a method of diagnosing a bone disease or disorder. The method employs a polynucleotide probe capable of hybridizing with the polynucleotide having the nucleic acid sequence set forth in SEQ ID NO: 1 to detect the presence or absence of an sFRP polynucleotide in a sample derived from a mammalian host.

A further embodiment relates to sFRP knockout animals in which expression of the sFRP mRNA, and consequently protein, is completely blocked. In particular, the present invention provides for sFRP-1 −/− animals.

### BRIEF DESCRIPTION OF THE DRAWINGS

FIGS. 1A-C are a set of graphs showing (A) cell proliferation of hOB-01-C1-PS-09 cells (B) the ability/enhancement in hOB-01-C1-PS-09 cells for vitamin D3—treatment to up-regulate alkaline phosphatase activity at 39° C., and (C) the inability of vitamin D3 to induce osteocalcin secretion from hOB cells at 34° C., as detailed in Example 1.

FIG. 2 depicts the results from Example 1 for the effect on intracellular cyclic adenosine monophosphate (cAMP) in response to increasing concentrations of PTH 1-34 in the hOB-01-C1-PS-09 cells.

FIG. 3 is a graph which depicts the effect of hOB-01-C1-PS-09 cells when treated with the synthetic glucocorticoid dexamethasone, which up-regulates alkaline phosphatase activity, as detailed in Example 1.

FIG. 4 shows an autoradiogram of a Northern blot of poly A+ RNA isolated from 23 different human tissues. In this experiment, both the excised hOB sFRP RADE gene fragment and a cloned beta-Actin cDNA were used as probes. The arrow points to the hOB sFRP mRNA that is highly expressed in heart and kidney, moderately expressed in placenta and uterus, expressed at lower levels in brain, pancreas and other tissues, but not expressed in thymus and lymphocytes.

FIG. 5 shows an autoradiogram of a Northern blot of poly A+ RNA isolated from SaOS-2 human osteosarcoma osteoblast-like cells and explant cultures of normal human osteoblasts (hOB) after treatment with control, PTH, PGE2 and TGF-β1 for 24 hr. In this experiment, both the full-length cloned hOB sFRP cDNA and a cloned GAPDH cDNA were used as probes. The results show that the SaOS-2 cells express low basal levels of hOB sFRP mRNA, which is not regulated by any of the test agents. In contrast, hOB cells express moderate levels of this message, which is up-regulated by treatment with PGE2. TaqMan quantitative RT-PCR analysis of hOB cells treated with PGE2 indicated that PGE2 upregulated sFRP message levels about 10-fold.

FIGS. 6A-C show the results of a cell viability experiment with hOB-03-C5 cells (which are proliferative) (A), hOB-03-CE6 cells (which are mature) (B), and hOB-01-C1 cells (which are preosteocytic) (C) using the Coulter cell counter. For these experiments, the cells were treated with either control or PGE2 (A & B) or control and TGF-β1 (C) in serum-free medium at 39° C. for 3 or 6 days. The results are presented as the % cell viability relative to the day 0 control (i.e., about 200,000 cells per well of a 6 well plate). The results of this experiment indicate that hOB cell viability declines over time in serum-free medium. In addition, for the hOB-03-C5 and hOB-03-CE6 cells, this rate of decline is accelerated by treatment with PGE2. This enhanced rate of cell death correlates with the upregulation of hOB sFRP mRNA levels in these cells following PGE2 treatment. In contrast, treatment of the hOB-01-C1 cells with TGF-β1, which downregulates hOB sFRP message levels, increased cell viability.

FIGS. 7A-C show that treatment of hOB-03-C5 cells with PGE2 induces apoptosis (programmed cell death). Apoptosis was measured by flow cytometry using annexin V-FITC. (A) shows that the number of viable cells, which do not stain with either annexin V or propidium iodide (a stain for necrotic cells), declines with increasing PGE2 concentrations. In contrast, (B) shows that the number of apoptotic cells, which stain with annexin V but not propidium iodide, increases with increasing PGE2 concentrations. Similarly, (C) shows that the number of necrotic cells, which stain with both annexin V and propidium iodide, increases with increasing PGE2 concentrations.

FIGS. 8A and B show the results of another cell viability experiment with the hOB-03-C5 cells (A) and hOB-03-CE6 cells (B) using the Coulter cell counter. The cells were co-treated with either vehicle control (i.e., 0.1% ethanol) or PGE2 in the absence or presence of sense (control) or antisense initiation-site directed phosphorothioate oligonucleotides to human sFRP-1. The results are presented as either the % cell viability relative to the day 0 control (i.e., about 200,000 cells per well of a 6 well plate) or as the % cell viability relative to the vehicle treated control. The results of this experiment indicate that co-treatment of the hOB cells with an antisense oligonucleotide to sFRP-1 reverses the ability of PGE2 to accelerate the rate of cell death, while co-treatment with the sense (control) oligonucleotide as no effect on this process.

FIGS. 9A and B show the results of a cell viability experiment with the hOB-01-C1-PS-09 cells using the Coulter cell counter (A). For these experiments, the cells were stably transfected with either an hOB sFRP cDNA (i.e., sFRP-1/FRP-1/SARP-2) mammalian expression plasmid or the empty vector (i.e., pcDNA3.1). The results are presented as the % cell viability of the day 0 control cells. The results of this experiment indicate that overexpression of hOB sFRP/sFRP-1/FRP-1/SARP-2 by the hOB cells accelerates the rate of cell death when compared to the empty vector, which has no effect on this process. (B) Results of a Northern hybridization of poly A+ RNA isolated from either the empty vector cells (V) or the sFRP-1 overexpressing cells (S). This analysis demonstrates that the sFRP-1 cells express substantially more sFRP-1/FRP-1/SARP-2 mRNA than the vector cells.

FIGS. 10A and B show the results of another cell viability experiment performed with the empty vector expressing hOB-01-C1-PS-09 cells (pcDNA3.1) and a subclone of the sFRP-1/FRP-1/SARP-2 overexpressing cells (SARP-2 Clone #1). (A) Results of a TaqMan quantitative RT-PCR analysis of RNA isolated from the cells. This analysis indicates that the SARP-2 Clone #1 cells express 50-60 times more human sFRP-1/FRP-1/SARP-2 mRNA than the empty vector expressing cells. Likewise, as shown in B, the SARP-2 Clone #1 cells die at a rate that is 3-times faster than the empty vector control cells using the Coulter cell counter to measure cell number.

FIG. 11 summarizes a screening paradigm for anabolic bone agents using the hOB cells and hOB sFRP/sFRP-1/FRP-1/SARP-2. This screening paradigm would identify compounds that regulate sFRP-1/FRP-1/SARP-2 function.

FIG. 12 depicts an example of a high-throughput screening (HTS) assay for compounds that inhibit sFRP-1/FRP-1/SARP-2 function in osteoblastic/osteocytic cells. The results show that the hOB-01-C1-PS-09 cells overexpressing sFRP-1/FRP-1/SARP-2 (SARP-2 #1 cells) die faster than the empty vector expressing cells (pcDNA3.1) using the CyQuant DNA fluorescence assay. Moreover, an antipeptide antiserum to sFRP-1/FRP-1/SARP-2 (SARP-2 AS) blocks cell death caused by overexpression of this gene.

FIG. 13 shows the results of a TaqMan real-time quantitative RT-PCR experiment with total RNA isolated from tissues obtained from 5 week old wild-type (+/+) C57BL/6J female and male mice. The results show that sFRP-1 mRNA, when normalized to the levels of 18S ribosomal RNA, is highly expressed in the kidney and ovary, but is expressed at lower levels in other tissues including the tibia. Results are presented as the mean±SEM, n=5.

FIGS. 14A and B show the results of a histological analysis of femur samples obtained from proximal femurs from 20-35 week old females (A) and males (B) shows delayed trabecular bone loss in sFRP-1−/− mice when compared to +/+ controls. Results are presented as the mean +/− SEM, n=8-15, *=p<0.01 versus +/+ controls.

FIGS. 15A-B show the results of a micro-computerized tomography (micro-CT) analysis of femurs obtained from 35 week old female (A) and male (B) wild-type (+/+) and knock-out (−/−) sFRP-1 mice. Quantification of double calcein labeling results demonstrate an increase in trabecular bone mineral apposition rate (MAR) in sFRP-1−/− females when compared to +/+ controls. Results are presented as the mean±SEM, n=8-10, *=p<0.001 versus +/+controls.

FIGS. 16A-B show the results of a peripheral quantitative computed tomography (pQCT) analysis of tibias obtained from 20-47 week old female (A) and male (B) wild-type (+/+) and knock-out (−/−) sFRP-1 mice. When compared to the +/+ control mice, the −/− mice exhibit increases in volumetric trabecular bone mineral density (vBMD) as determined by this method.

FIGS. 17A and B show the results of an in vitro differentiation experiment with bone marrow isolated from femurs and tibias obtained from 25-26 week old female wild-type (+/+) and knock-out (−/−) sFRP-1 mice. (A) Quantification of alkaline phosphatase (ALP) positive cells after stimulation of osteoblast differentiation for 21 days with ascorbate-2-phosphate (Asc-2-P), β-glycerol phosphate (β-GP), and dexamethasone (Dex). Results show increased differentiation in bone marrow cells from 25-26 week old female sFRP-1−/− mice when compared to +/+ controls. (B) Quantification of tartrate-resistant acid phosphatase (TRAP) positive cells after stimulation of osteoclast differentiation with soluble recombinant human receptor activator of nuclear factor-κB ligand (srhRANKL) and recombinant murine monocyte-colony stimulating factor (rmM-CSF). Results show increased differentiation in bone marrow cells from 25-26 week old female sFRP-1−/− mice when compared to +/+ controls. Data are presented as the mean +/−SD, n=4 wells, *=p<0.05 versus +/+ controls.

FIGS. 18A and B show that deletion of sFRP-1 in mice results in an increase in bone marrow-derived osteoprogenitor and calvarial-derived osteoblast proliferation in vitro. (A) Measurement of [3H]-thymidine incorporation in bone marrow progenitor cells after 7 days in culture. Results show increased DNA synthesis in bone marrow cells from 32-week-old female sFRP-1−/− mice when compared to +/+ controls. (B) Measurement of [3H]-thymidine incorporation in primary neonatal calvarial-derived osteoblast cultures. Results show increased DNA synthesis in osteoblasts from neonatal sFRP-1−/− mice when compared to +/+ controls. Data are presented as the mean±SD, n=3 wells, *=p<0.05 versus +/+ controls.

### DETAILED DESCRIPTION

The present invention relates to a gene whose expression is regulated by osteogenic or bone-forming agents in three different human osteoblast (hOB) cell lines in vitro. In one aspect of the present invention, the expression of this gene is upregulated during hOB differentiation, suggesting it may be involved in the bone formation process. DNA sequence analysis indicated that this gene fragment shared significant sequence identity to a mouse cDNA called secreted frizzled-related protein (sFRP)-1 (Rattner et al., Proc. Natl. Acad. Sci. USA 1997, 94:2859-2863). Subsequent cDNA cloning and additional sequence analysis indicated that the gene of the present invention, which is referred to as the hOB sFRP, was, except for a one amino acid difference at position 174, identical to human sFRP-1/FRP-1/SARP-2 (Finch et al., Proc. Natl. Acad. Sci. USA 1997, 94:6770-6775; Melkonyan et al., Proc. Natl. Acad. Sci. USA 1997, 94:13636-13641). Thus, this gene and its product are also herein referred to as sFRP-1.

Development of a sFRP-1 −/− mouse line provided an experimental approach to address the contribution of Wnt signaling to bone biology and to determine if sFRP-1 regulates osteoblast and osteocyte viability in vivo. These mice show that deletion of sFRP-1 not only reduces osteoblast and osteocyte apoptosis, but also potentiates osteoprogenitor cell differentiation and increases trabecular bone formation. Unexpectedly, targeted ablation of sFRP-1 delays the onset of age-dependent trabecular bone loss, while having little effect on fertility, body weight, blood and urine chemistries, non-skeletal organs or cortical bone. These results indicate that sFRP-1 not only plays a role in the attainment of peak bone mass, but also regulates senile bone loss.

Thus, characterization of this activity of the hOB sFRP/sFRP-1 establishes various approaches to treating osteoporosis, including use of the protein as a drug target and drug screen for identifying anabolic bone agents and other agents involved in bone formation, repair, and remodeling, and use of nucleic acids specific for sFRP/sFRP-1 to inhibit expression of the protein.

The term “bone formation” is the process of bone synthesis and mineralization. The osteoblast cell modulates the process.

The term “bone growth” is the process of skeletal expansion. This process occurs by one of two ways: (1) intramembraneous bone formation arises directly from mesenchymal or bone marrow cells; (2) longitudinal or endochondral bone formation arises where bone forms from cartilage.

The term “osteogenesis” is synonymous with the term bone formation, defined above.

The terms “secreted frizzled related proteins” or “sFRP” is a secreted receptor of the Wnt signaling pathway and exhibits a number of characteristics that make it a useful tool for studying cell growth and differentiation. The frizzled like gene family encodes cell membrane proteins having transmembrane domains with unknown functions.

sFRP-1 is one preferred member of the sFRP family of proteins. sFRP-1 can have the amino acid sequence set forth in SEQ ID NO: 2, as encoded for by SEQ ID NO: 1. SEQ ID NO: 2 contains one amino acid different from the published sequence for human sFRP-1: alanine 174 instead of proline at this position (Melkonyan et al., Proc. Natl. Acad. Sci. USA 1997, 94:13636-13641). SEQ ID NO: 2 also differs by one amino acid from another reported sFRP-1 sequence (GenBank™ Accession No. NP_{—}003003; SEQ ID NO: 7) because it has 314 amino acids, while GenBank™ Accession No. NP_{—}003003 has 313 amino acids (this difference is due to an additional alanine residue at position 13 in SEQ ID NO: 2). The present invention encompasses compositions comprising and methods using all sFRP-1 proteins and polypeptides (and fragments thereof), and nucleotides encoding these polypeptides, including those shown in SEQ ID NO: 2, polypeptides being about at least 90% identical to SEQ ID NO: 2 (encoded for by polynucleotides having a sequence about at least 85% identical to SEQ ID NO: 1), GenBank™ Accession No. NP_{—}003003, the sFRP-1 as reported in Melkonyan et al. (supra), and muteins and allelic variants of these polynucleotides and polypeptides.

The terms “secreted apoptosis related protein” or “SARP” are synonymous with the term secreted frizzled related proteins or sFRPs defined above. The terms hOB sFRP, FRP-1, FrzA and SARP-2 are synonymous with the term sFRP-1.

The terms “proteins”, “peptides” and “polypeptides” are used interchangeably and are intended to include purified and recombinantly produced sFRP molecules containing amino acids linearly coupled through peptide bonds. The amino acids of this invention can be in the L or D form so long as the biological activity of the polypeptide is maintained. The sFRP proteins of this invention may also include proteins that are post-translationally modified by reactions that include glycosylation, acetylation and phosphorylation. Such polypeptides also include analogs, alleles and allelic variants that can contain amino acid derivatives or non-amino acid moieties that do not affect the biological or functional activity of the sFRP protein as compared to wild-type or naturally occurring protein. The term “amino acid” refers both to the naturally occurring amino acids and their derivatives, such as TyrMe and PheCl, as well as other moieties characterized by the presence of both an available carboxyl group and an amine group. Non-amino acid moieties that can be contained in such polypeptides include, for example, amino acid mimicking structures. Mimicking structures are those structures that exhibit substantially the same spatial arrangement of functional groups as amino acids but do not necessarily have both the amino and carboxyl groups characteristic of amino acids.

“Muteins” are sFRP proteins or polypeptides that have minor changes in amino acid sequence caused, for example, by site-specific mutagenesis or other manipulations; by errors in transcription or translation; or which are prepared synthetically by rational design. These minor alterations result in amino acid sequences, which may alter a biological activity or other characteristics of the protein or polypeptide compared to wild-type or naturally occurring polypeptide or protein. Examples of muteins include the sFRP-1 of SEQ ID NO: 2 described herein.

“Isolated,” when referring to a sFRP nucleic acid molecule, means separated from other cellular components normally associated with native or wild-type sFRP DNA or RNA intracellularly.

“Purified” when referring to a sFRP protein or polypeptide, is distinguishable from native or naturally occurring proteins or polypeptides because they exist in a purified state. These “purified” sFRP proteins or polypeptides, or any of the intended variations as described herein, shall mean that the compound or molecule is substantially free of contaminants normally associated with the compound in its native or natural environment. The terms “substantially pure” and “isolated” are not intended to exclude mixtures of polynucleotides or polypeptides with substances that are not associated with the polynucleotides or polypeptides in nature.

“Native” sFRP polypeptides, proteins, or nucleic acid molecules refer to those sFRP recovered from a source occurring in nature or “wild-type.”

The term “nucleic acid” as it relates to the sFRP described herein means single and double-stranded DNA, cDNA, genome-derived DNA, and RNA, as well as the positive and negative strand of the nucleic acid that are complements of each other, including anti-sense RNA. A “nucleic acid molecule” is a term used interchangeably with “polynucleotide” and each refers to a polymeric form of nucleotides of any length, either ribonucleotides or deoxyribonucleotides, or analogs thereof. It also includes known types of modifications, for example labels which are known in the art (e.g., Sambrook et al., (1989) infra.), methylation, “caps”, substitution of one or more of the naturally occurring nucleotides with an analog, internucleotide modifications such as, for example, those with uncharged linkages (e.g., methyl carbamate, etc.), those containing pendant moieties, such as for example, proteins (including, e.g., nuclease, toxins, antibodies, signal peptides, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. The polynucleotide can be chemically or biochemically modified or contain non-natural or derivatized nucleotide bases. The nucleotides may be complementary to the mRNA encoding the polypeptides. These complementary nucleotides include, but are not limited to, nucleotides capable of forming triple helices and antisense nucleotides. Recombinant polynucleotides comprising sequences otherwise not naturally occurring are also provided by this invention, as are alterations of wild-type polypeptide sequences, including but not limited to, those due to deletion, insertion, substitution of one or more nucleotides or by fusion to other polynucleotide sequences.

An sFRP polynucleotide is said to “encode” a sFRP polypeptide if, in its native state or when manipulated by methods well-known to those skilled in the art, it can be transcribed and/or translated to produce a polypeptide or mature protein. Thus, the term polynucleotide shall include, in addition to coding sequences, processing sequences and other sequences that do not code for amino acids of the mature protein. The anti-sense strand of such a polynucleotide is also said to encode the sequence.

The term “recombinant” polynucleotide or DNA refers to a polynucleotide that is made by the combination of two otherwise separated segments of sequence accomplished by the artificial manipulation of isolated segments of DNA by genetic engineering techniques or by chemical synthesis. In so doing, one may join together DNA segments of desired functions to generate a desired combination of functions.

An “analog” of a sFRP DNA, RNA or a polynucleotide, refers to a macromolecule resembling naturally occurring polynucleotides in form and/or function (particularly in the ability to engage in sequence-specific hydrogen bonding to base pairs on a complementary polynucleotide sequence) but which differs from DNA or RNA in, for example, the possession of an unusual or non-natural base or an altered backbone. See for example, Uhlmann et al. Chemical Reviews 1990, 90:543-584.

“Hybridization” refers to hybridization reactions that can be performed under conditions of different “stringency”. Conditions that increase the stringency of a hybridization reaction are widely known and published in the art: see, for example, Sambrook et al., infra. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%, incubation times from 5 minutes to 24 hours and washes of increasing duration, increasing frequency, or decreasing buffer concentrations.

“Tₘ” is the temperature in degrees Centigrade at which 50% of a polynucleotide duplex made of complementary strands hydrogen bonded in an antiparallel direction by Watson-Crick base paring dissociates into single strands under the conditions of the experiment. Tₘ may be predicted according to standard formulas, for example:

Tₘ=81.5+16.6 log[Na⁺]+0.41(% G/C)−0.61(% F)−600/L

where Na⁺ is the cation concentration (usually sodium ion) in mol/L; (% G/C) is the number of G and C residues as a percentage of total residues in the duplex; (% F) is the percent formamide in solution (wt/vol); and L is the number of nucleotides in each strand of the duplex.

A “stable duplex” of polynucleotides, or a “stable complex” formed between any two or more components in a biochemical reaction, refers to a duplex or complex that is sufficiently long lasting to persist between the formation of the duplex or complex, and its subsequent detection. The duplex or complex must be able to withstand whatever conditions exist or are introduced between the moment of formation and the moment of detection, these conditions being a function of the assay or reaction which is being performed. Intervening conditions which may optionally be present and which may dissociate a duplex or complex include washing, heating, adding additional solutes or solvents to the reaction mixture (such as denaturants), and competing with additional reacting species. Stable duplexes or complexes may be irreversible or reversible, but must meet the other requirements of this definition. Thus, a transient complex may form in a reaction mixture, but it does not constitute a stable complex if it dissociates spontaneously or as a result of a newly imposed condition or manipulation introduced before detection.

When stable duplexes form in an antiparallel configuration between two single-stranded polynucleotides, particularly under conditions of high stringency, the strands are essentially “complementary”. A double-stranded polynucleotide can be “complementary” to another polynucleotide, if a stable duplex can form between one of the strands of the first polynucleotide and the second. A complementary sequence predicted from the sequence of single stranded polynucleotide is the optimum sequence of standard nucleotides expected to form hydrogen bonding with the single-stranded polynucleotide according to generally accepted base-pairing rules.

A “sense” strand and an “antisense” strand when used in the same context refer to single-stranded sFRP polynucleotides which are complementary to each other. They may be opposing strands of a double-stranded polynucleotide, or one strand may be predicted from the other according to generally accepted base-pairing rules. Unless otherwise specified or implied, the assignment of one or the other strand as “sense” or “antisense” is arbitrary.

A linear sequence of sFRP nucleotides is “identical” to another linear sequence, if the order of nucleotides in each sequence is the same, and occurs without substitution, deletion, or material substitution. It is understood that purine and pyrimidine nitrogenous bases with similar structures can be functionally equivalent in terms of Watson-Crick base-pairing; and the inter-substitution of like nitrogenous bases, particularly uracil and thymine, or the modification of nitrogenous bases, such as by methylation, does not constitute a material substitution. An RNA and a DNA polynucleotide have identical sequences when the sequence for the RNA reflects the order of nitrogenous bases in the polyribonucleotide, the sequence for the DNA reflects the order of nitrogenous bases in the polydeoxyribonucleotide, and the two sequences satisfy the other requirements of this definition. Where at least one of the sequences is a degenerate oligonucleotide comprising an ambiguous residue, the two sequences are identical if at least one of the alternative forms of the degenerate oligonucleotide is identical to the sequence with which it is being compared. For example, AYAAA is identical to ATAAA, if AYAAA is a mixture of ATAAA and ACAAA.

When comparison is made between polynucleotides, it is implicitly understood that complementary strands are easily generated, and the sense or antisense strand is selected or predicted that maximizes the degree of identity between the polynucleotides being compared. For example, where one or both of the polynucleotides being compared is double-stranded, the sequences are identical if one strand of the first polynucleotide is identical with one strand of the second polynucleotide. Similarly, when a polynucleotide probe is described as identical to its target, it is understood that it is the complementary strand of the target that participates in the hybridization reaction between the probe and the target.

A linear sequence of nucleotides is “essentially identical” or the “equivalent” to another linear sequence, if both sequences are capable of hybridizing to form duplexes with the same complementary polynucleotide. It should be understood, although not always explicitly stated, that Applicants refer to a specific nucleic acid molecule, its equivalents are also intended. Sequences that hybridize under conditions of greater stringency are more preferred. It is understood that hybridization reactions can accommodate insertions, deletions, and substitutions in the nucleotide sequence. Thus, linear sequences of nucleotides can be essentially identical even if some of the nucleotide residues do not precisely correspond or align. Sequences that correspond or align more closely to the invention disclosed herein are comparably more preferred. Generally, a polynucleotide region of about 25 residues is essentially identical to another region, if the sequences are at least about 85% identical; more preferably, they are at least about 90% identical; more preferably, they are at least about 95% identical; still more preferably, the sequences are 100% identical. A polynucleotide region of 40 residues or more will be essentially identical to another region, after alignment of homologous portions if the sequences are at least about 85% identical; they are at least about 90% identical; more preferably they are 95% identical, and still more preferably, the sequences are 100% identical.

In determining whether polynucleotide sequences are essentially identical, a sequence that preserves the functionality of the polynucleotide with which it is being compared is particularly preferred. Functionality can be determined by different parameters. For example, if the polynucleotide is to be used in reactions that involve hybridizing with another polynucleotide, then preferred sequences are those which hybridize to the same target under similar conditions. In general, the Tₘ of a DNA duplex decreases by about 10° C. for every 1% decrease in sequence identity for duplexes of 200 or more residues; or by about 50° C. for duplexes of less than 40 residues, depending on the position of the mismatched residues (see, e.g. Meinkoth and Wahl, Analytical Biochemistry 1984, 138:267-284). Essentially identical or equivalent sequences of about 100 residues will generally form a stable duplex with each other's respective complementary sequence at about 20° C. less than Tₘ; preferably, they will form a stable duplex at about 15° C. less; more preferably, they will form a stable duplex at about 10° C. less; even more preferably, they will form a stable duplex at about 5° C. less; still more preferably, they will form a stable duplex at about Tₘ. In another example, if the polypeptide encoded by the polynucleotide is an important part of its functionality, then preferred sequences are those which encode identical or essentially identical polypeptides. Thus, nucleotide differences which cause a conservative amino acid substitution are preferred over those which can cause a non-conservative amino acid substitution are preferred over those which cause a non-conservative substitution, nucleotide differences which do not alter the amino acid sequence are more preferred, while identical nucleotides are even more preferred. Insertions or deletions in the polynucleotide that result in insertions or deletions in the polypeptide are preferred over those that result in the down-stream coding regions being rendered out of phase; polynucleotide sequences comprising no insertions or deletions are even more preferred. The relative importance of hybridization properties and the encoded polypeptide sequence of a polynucleotide depends on the application of the invention.

A polynucleotide has the same characteristics or is the equivalent of another polynucleotide if both are capable of forming a stable duplex with a particular third polynucleotide under similar conditions of maximal stringency. Preferably, in addition to similar hybridization properties, the polynucleotides also encode essentially identical polypeptides.

“Conserved” residues of a polynucleotide sequence are those residues that occur unaltered in the same position of two or more related sequences being compared. Residues that are relatively conserved are those that are conserved amongst more related sequences than residues appearing elsewhere in the sequences.

“Related” polynucleotides that share a significant proportion of identical residues.

As used herein, a “degenerate” oligonucleotide sequence is a designed sequence derived from at least two related originating polynucleotide sequences as follows: the residues that are conserved in the originating sequences are preserved in the degenerate sequence, while residues that are not conserved in the originating sequences may be provided as several alternatives in the degenerate sequence. For example, the degenerate sequence AYASA may be assigned from originating sequences ATACA and ACAGA, where Y is C or T and S is C or G. Y and S are examples of “ambiguous” residues. A degenerate segment is a segment of a polynucleotide containing a degenerate sequence.

It is understood that a synthetic oligonucleotide comprising a degenerate sequence is actually a mixture of closely related oligonucleotides sharing an identical sequence, except at the ambiguous positions. Such an oligonucleotide is usually synthesized as a mixture of all possible combinations of nucleotides at the ambiguous positions. Each of the oligonucleotides in the mixture is referred to as an “alternative form”.

A polynucleotide “fragment” or “insert” as used herein generally represents a sub-region of the full-length form, but the entire full-length polynucleotide may also be included.

Different polynucleotides “correspond” to each other if one is ultimately derived from another. For example, messenger RNA corresponds to the gene from which it is transcribed. cDNA corresponds to the RNA from which it has been produced, such as by a reverse transcription reaction, or by chemical synthesis of a DNA based upon knowledge of the RNA sequence or the coding sequence of genomic DNA. cDNA also corresponds to the coding sequence of the gene that encodes the RNA. Polynucleotides also “correspond” to each other if they serve a similar function, such as encoding a related polypeptide, in different species, strains or variants that are being compared.

A “probe” when used in the context of sFRP polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is an oligonucleotide, generally with a free 3′-OH group, that binds to a target potentially present in a sample of interest by hybridizing with the target, and thereafter promotes polymerization of a polynucleotide complementary to the target.

Processes of producing replicate copies of the same polynucleotide, such as PCR or gene cloning, are collectively referred to herein as “amplification” or “replication”. For example, single or double-stranded DNA may be replicated to form another DNA with the same sequence. RNA may be replicated, for example, by an RNA-directed RNA polymerase, or by reverse-transcribing the DNA and then performing a PCR. In the latter case, the amplified copy of the RNA is a DNA with the identical sequence.

Elements within a gene include but are not limited to promoter regions, enhancer regions, repressor binding regions, transcription initiation sites, ribosome binding sites, translation initiation sites, protein encoding regions, introns and exons, and termination sites for transcription and translation. An “antisense” copy of a particular polynucleotide refers to a complementary sequence that is capable of hydrogen bonding to the polynucleotide and can therefor, be capable of modulating expression of the polynucleotide. These are DNA, RNA or analogs thereof, including analogs having altered backbones, as described above. The polynucleotide to which the antisense copy binds may be in single-stranded form or in double-stranded form.

As used herein, the term “operatively linked” means that the DNA molecule is positioned relative to the necessary regulation sequences, e.g., a promoter or enhancer, such that the promoter will direct transcription of RNA off the DNA molecule in a stable or transient manner.

“Vector” means a self-replicating nucleic acid molecule that transfers an inserted nucleic acid molecule into and/or between host cells. The term is intended to include vectors that function primarily for the replication of nucleic acid and expression vectors that function for transcription and/or translation of the DNA or RNA. Also intended are vectors that provide more than one of the above functions.

“Host cell” is intended to include any individual cell or cell culture that can be or have been recipients for vectors or the incorporation of exogenous nucleic acid molecules and/or proteins. It also is intended to include progeny of a single cell, and the progeny may not necessarily be completely identical (in morphology or in genomic or total DNA complement) to the original parent cell due to natural, accidental, or deliberate mutation.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody (as defined above) and its binding partner or ligand.

A “suitable cell” for the purposes of this invention is one that includes but is not limited to a cell expressing the sFRP, e.g., a bone marrow cell, preferentially an hOB cell.

A “biological equivalent” of a nucleic acid molecule is defined herein as one possessing essential identity with the reference nucleic acid molecule. A fragment of the reference nucleic acid molecule is one example of a biological equivalent.

A “biological equivalent of an sFRP polypeptide or protein” is one that retains the same characteristic as the reference protein or polypeptide. It also includes fragments of the reference protein or polypeptide.

The sFRP proteins and polypeptides also can be obtained by chemical synthesis using a commercially available automated peptide synthesizer such as those manufactured by Applied Biosystems, Inc., Model 430A or 431A, Foster City, Calif. and the amino acid sequence provided in SEQ ID NO: 2. The synthesized protein or polypeptide can be precipitated and further purified, for example by high performance liquid chromatography (HPLC). Accordingly, this invention also provides a process for chemically synthesizing the proteins of this invention by providing the sequence of the protein (e.g., SEQ ID NO: 2) and reagents, such as amino acids and enzymes and linking together the amino acids in the proper orientation and linear sequence.

Alternatively, the proteins and polypeptides can be obtained by well-known recombinant methods as described, for example, in Sambrook et al., Molecular Cloning: A Laboratory Manual 2d ed. (Cold Spring Harbor Laboratory (1989)) using, for example, the host cell and vector systems described and exemplified below. This invention further provides a process for producing a sFRP, analog, mutein or fragment thereof, by growing a host cell containing a nucleic acid molecule encoding the desired protein, the nucleic acid being operatively linked to a promoter of RNA transcription. The desired protein may be introduced into the host cell by use of a gene construct which contains a promoter and termination sequence for the nucleic acid sequence of the desired protein. The host cell is grown under suitable conditions such that the nucleic acid is transcribed and translated into protein. In a separate embodiment, the protein is further purified.

The proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to nonspecifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

### sFRP Knockout Mammals

The present invention also provides for mammals in which expression of the sFRP has been completely abrogated or decreased due to “knocking-out” one or both copies of the sFRP gene. In particular, the present invention provides for mammals, specifically mice, in which the sFRP-1 gene has been knocked-out and thus, sFRP-1 expression has been completely eliminated. Such sFRP-1 −/− mice do not express sFRP-1 mRNA (as can be determined by Northern blot analysis and RT-PCR) and thus do not express any sFRP-1 protein (as can be determined by Western blot analysis). Further phenotypic characteristics of such sFRP-1 −/− animals are demonstrated in detail in Examples 15-20 of this application and include, but are not limited to, increased trabecular bone formation, enhanced osteoprogenitor differentiation, decreased levels of osteoblast and osteocyte apoptosis, and increased bone marrow-derived osteoprogenitor cell and calvarial-derived osteoblast proliferation. The sFRP-1 knockout mice do not have altered bone resorption or skeletal development

A “knockout mammal” is a mammal (e.g., mouse) that contains within its genome a specific gene that has been inactivated by the method of gene targeting (see, e.g., U.S. Pat. Nos. 5,777,195 and 5,616,491). A knockout mammal includes both a heterozygote knockout (i.e., one defective allele and one wild type allele) and a homozygous mutant. Preparation of a knockout mammal requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou, et al. (Genes and Development 1995, 9:2623 34) describes PPCA knock out mice.

The term “knockout” refers to partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise (1) a full or partial sequence of one or more exons and/or introns of the gene to be suppressed, (2) a full or partial promoter sequence of the gene to be suppressed, or (3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

Generally, for homologous recombination, the DNA will be at least about 1 kilobase (kb) in length and preferably 3-4 kb in length, thereby providing sufficient complementary sequence for recombination when the knockout construct is introduced into the genomic DNA of the ES cell.

Included within the scope of this invention is a mammal in which two or more genes have been knocked out or knocked in, or both. Such mammals can be generated by repeating the procedures set forth herein for generating each knockout construct, or by breeding to mammals, each with a single gene knocked out, to each other, and screening for those with the double knockout genotype.

Regulated knockout animals can be prepared using various systems, such as the tet-repressor system (see U.S. Pat. No. 5,654,168) or the Cre-Lox system (see U.S. Pat. No. 4,959,317 and No. 5,801,030).

### Therapeutic Applications for sFRPs/SARPs

Although the sFRPs/SARPs gene family has only recently been discovered and there is still much to learn about its biology, there are nevertheless several potential therapeutic applications for these proteins. Since Wnts have been implicated as proto-oncogenes, sFRPs/SARPs may serve as tumor suppressors due to their ability to antagonize Wnt activity. These proteins may also be utilized in tissue regeneration. For example, since FrzB-1 stimulated ectopic chondrogenic activity in vivo, it could be used to accelerate fracture repair or the healing of joints after hip and knee replacement (see, International Patent Publication No. WO 98/16641 A1). Finally, since sFRPs/SARPs appear to control apoptosis, these proteins could also be utilized to treat a variety of degenerative diseases including neurodegeneration, myodegeneration and osteodegeneration disorders.

In preferred embodiments, pharmaceutical compositions of the invention are useful for treating osteodegeneration disorders such as osteoporosis and the bone resportive disease Paget's disease. For instance, the examples, infra, demonstrate that when sFRP-1 expression and/or activity are abolished in vivo (e.g., in transgenic mice) bone density is increased, resulting in a delay of age-dependent bone loss. These effects correlate generally with an increased rate of bone formation, a decrease in osteoblast and osteoclast apoptosis, and an increase in osteoblast differentiation. Disruption of the fine balance between the differentiation of new osteoclast and osteoblast cells, and the timing of cell death by apoptosis is thought to be an important mechanism behind bone loss disorders such as osteogenesis. Thus, therapeutic agents that alter the prevalence of apoptosis in osteoblasts and/or osteoclasts are useful and desirable to correct the imbalance in cell numbers that is the basis of diminished bone mass and increased risk of fractures in osteoporosis. For review, see, Manolagas, Endocrine Reviews 2002, 21:115-137; and Weinstein and Manolagas, Am. J. Med. 2000, 108:153-164. The data presented here therefore demonstrate that compositions and methods of the invention which target and/or inhibit sFRP-1 are useful, not only to treat an existing osteodegeneration disorder, but also in preventive therapies to inhibit or prevent bone loss in an individual.

For example, in one preferred embodiment the pharmaceutical compositions and methods of this invention may be used to prevent an osteodegenerative disorder, e.g., in an individual who may not have a bone degeneration disorder but who has or is suspecting of being susceptible to such a disorder. In a particularly preferred embodiment, the pharmaceutical compositions and methods of the invention can be used to prevent Type II or “senile” osteoporosis. As a particular example, and not by way of limitation, a therapy or pharmaceutical composition of the invention may be administered to a juvenile, adolescent or young adult.

Moreover, altering the activity of sFRP-1 does not produce any other significant side effects, e.g., on cortical bone and non-skeletal tissues, in body or organ weight; serum calcium, phosphorus, bone-alkaline phosphatase or osteocalcin levels; urinary deoxy-pyridinoline cross-link levels; total body BMD, bone mineral content and percentage body fat; or cortical BMD. Even though the inhibition of sFRP-1 may increase bone density, it does not alter skeletal development. Consequently, therapeutic methods and compositions that specifically inhibit sFRP-1 activity and/or expression are expected to have very few or even no detrimental side effects.

In preferred embodiments, therefore, the compositions and methods of the invention produce very high levels of SRFP inhibition without causing undue or detrimental side effects. For example, preferred pharmaceutical compositions and methods of the invention inhibit sFRP/SARP expression or activity by at least 10%, more preferably by 20% and most preferably by 30%, 40%, 50%, 60%, 70%, 80%, 90% or 95%. Indeed, particularly preferred methods and compositions of the invention will completely or almost completely abolish sFRP expression and/or activity in an individual (i.e., by 100%). In preferred embodiments, the methods and compositions of the invention inhibit the particular sFRP/SARP gene produce referred to here as sFRP-1.

Alternatively the methods and compositions of the present invention can be used to treat diseases, such as osteopetrosis and osteoschlerosis, that are the result of aberrant bone formation or abnormal increases in bone formation. Such diseases are treated by disrupting or decreasing sFRP expression or sFRP activity by means of, for example, antisense sFRP nucleotides, siRNAs or shRNAs that inhibit sFRP expression, or small molecule inhibitors that disrupt or decrease sFRP activity and/or expression.

### Pharmaceutical Compositions

A “pharmaceutical composition” is intended to include the combination of sFRP, particularly sFRP-1, or antibodies against an sFRP, antisense nucleotides, ribozymes, siRNAs and shRNAs for decreasing or blocking sFRP expression or activity, as the active agent with a carrier, inert or active, making the composition suitable for diagnostic or therapeutic use in vitro, in vivo or ex vivo.

This invention also provides compositions containing any of the above-mentioned proteins, muteins, fragments, antibodies, nucleic acid molecules encoding such proteins, muteins, antibodies or fragments thereof, as well as vectors and host cells that express such nucleic acid molecules, and an acceptable solid or liquid, carrier buffer, or diluent. An effective amount of one or more active ingredient is used which is sufficient to accomplish the desired regulatory effect on a bone-forming activity or apoptosis activity. An effective amount can be determined by conventional dose-response curves for the desired activity. When the compositions are used pharmaceutically, they are combined with a “pharmaceutically acceptable carrier” for diagnostic and therapeutic use. The formulation of such compositions is well known to persons skilled in this field. Pharmaceutical compositions of the invention may comprise one or more additional active components and, preferably, include a pharmaceutically acceptable carrier. The additional active component may be provided to work in combination with an active based on a one or more sFRPs, as described above. In alternative embodiments, the additional active is added because it works on the same disease or disorder as sFRPs but by a different mode of action from those actives based on sFRPs, or the additional active may work on other diseases or disorders present in a human or animal. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like with which the compound is administered. The term “pharmaceutically acceptable carrier” refers to a carrier that does not cause an allergic reaction or other untoward effect in patients to whom it is administered. Preferably, as used herein, the term “pharmaceutically acceptable” means approved by a regulatory agency of the Federal or state government or listed in the U.S. Pharmacopeia or other generally recognized pharmacopeia for use in animals, and more particularly, in humans. Suitable pharmaceutically acceptable carriers include, for example, one or more of water, saline, phosphate buffered saline, dextrose, glycerol, ethanol and the like, as well as combinations thereof. Water or aqueous solution saline solutions and aqueous dextrose and glycerol solutions are preferably employed as carriers, particularly for injectable solutions. Pharmaceutically acceptable carriers may further comprise minor amounts of auxiliary substances such as wetting or emulsifying agents, preservatives or buffers, which enhance the shelf life or effectiveness of one or more of the active components of the composition. The use of such media and agents for pharmaceutically active substances is well known in the art and suitable pharmaceutical carriers are described in “Remington's Pharmaceutical Sciences” by E. W. Martin. Except insofar as any conventional media or agent is incompatible with the active ingredient, use thereof in immunogenic compositions of the present invention is contemplated.

These compositions also can be used for the preparation of medicaments for the diagnosis and treatment of pathologies associated with neurodegenerative (i.e., Huntington's disease, Alzheimer's disease, spinal cord injuries), myodegenerative (i.e., muscular dsytrophy, myasthenia gravis, myotonic myopathies) and osteodegenerative disorders (i.e., osteoporosis). These compositions can also be used for the preparation for medicaments for the diagnosis and treatment of diseases such as Paget's disease, osteoschlerosis, osteogenesis imperfecta, fibrous dysplasia, hypophosphatasia and osteopetrosis.

In certain embodiments, antibodies that bind all or a portion of an sFRP protein are employed in the composition to treat of any of the above diseases or disorders. Preferred antibodies for in vivo use recognize a topological or conformational epitope present on the native sFRP molecule. Such antibodies may or may not recognize denatured sFRP or sFRP fragments. Polyclonal and monoclonal antibodies can be prepared by conventional methods, e.g., by immunization with sFRP protein. Alternatively, an antibody is raised against an amino acid sequence (a) that is specific to a sFRP polypeptide/protein (or proteins) and (b) that is also more likely to be antigenic. One can select a sequence specific for an sFRP protein by performing sequence analysis and using any conventional programs for sequence alignment and sequence comparisons. An amino acid sequence that is hydrophilic at one or more ends, preferably at both ends, is generally preferred for raising antibodies. In addition to employing amino acids that are hydrophilic, in preferred embodiments the hydrophilic amino acids are also basic (non-acidic). One can also employ any amino acid that increases antigenicity. For example, often prolines are employed in the center portion of the sequence. Antigenicity can be measured by an increase in decrease in the amount of antibody that is produced when generating antibodies against an initial test sequence, which is specific an sFRP protein(s). In certain embodiments of the present invention, the antibody is raised against a sequence comprising at least 8 consecutive amino acids of an sFRP protein(s), and preferably a sequence comprising at least 10 consecutive amino acids of an sFRP protein(s). In further preferred embodiments, the antibody is raised against amino acid sequence comprising about 15 to about 30 amino acids. In preferred embodiments, the antibody is raised against a sequence comprising at amino acids 217-231 of a sFRP-1 protein, a polypeptide have the amino acid sequence set forth in SEQ ID NO: 2, or sequence variations thereof.

The compositions of the present invention can be administered to an individual in need of facilitated neural, muscle cartilage and bone growth by numerous routes, including but not limited to intravenous, subcutaneous, intramuscular, intrathecal, intracranial and topical. The composition may be administered directly to an organ or to organ cells by in vivo or ex vivo methods.

These compositions may be in soluble or microparticular form, or may be incorporated into microspheres or microvesicles, including micelles and liposomes.

The pharmaceutical compositions of the present invention comprising nucleic acids can be used to “knock down” or “knock out” the expression of the corresponding genes in a cell or tissue (e.g., in an animal model or in cultured cells) by using their sequences to design antisense oligonucleotides, RNA interference (RNAi) molecules, ribozymes, nucleic acid molecules to be used in triplex helix formation, etc. Preferred methods to inhibit the expression of sFRPs are described below.

### RNA Interference (RNAi)

RNA interference (RNAi) is a process of sequence-specific post-transcriptional gene silencing by which double-stranded RNA (dsRNA) homologous to a target locus can specifically inactivate gene function in plants, fungi, invertebrates, and mammalian systems (Hammond et al., Nature Genet. 2001, 2:110-119; Sharp, Genes Dev. 1999, 13:139-141). This dsRNA-induced gene silencing is mediated by 21- and 22-nucleotide double-stranded small interfering RNAs (siRNAs) generated from longer dsRNAs by ribonuclease III cleavage (Bernstein et al., Nature 2001, 409:363-366 and Elbashir et al., Genes Dev. 2001, 15:188-200). RNAi-mediated gene silencing is thought to occur via sequence-specific mRNA degradation, where sequence specificity is determined by the interaction of an siRNA with its complementary sequence within a target mRNA (see, e.g., Tuschl, Chem. Biochem. 2001, 2:239-245).

For mammalian systems, RNAi commonly involves the use of dsRNAs that are greater than 500 bp, however, it can be also activated by introduction of either siRNAs (Elbashir, et al., Nature 2001, 411:494-498) or short hairpin RNAs (shRNAs) bearing a fold back stem-loop structure (Paddison et al., Genes Dev. 2002, 16:948-958; Sui et al., Proc. Natl. Acad. Sci. USA 2002, 99:5515-5520; Brummelkamp et al., Science 2002, 296:550-553; Paul et al., Nature Biotechnol. 2002, 20:505-508).

The siRNAs to be used in the methods of the present invention are short double-stranded nucleic acid duplexes comprising annealed complementary single stranded nucleic acid molecules. In preferred embodiments, the siRNAs are short dsRNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule.

Preferably, each single stranded nucleic acid molecule of the siRNA duplex is of from about 21 nucleotides to about 27 nucleotides in length. In preferred embodiments, duplexed siRNAs have a 2 or 3 nucleotide 3′ overhang on each strand of the duplex. In preferred embodiments, siRNAs have 5′-phosphate and 3′-hydroxyl groups.

According to the present invention, siRNAs may be introduced to a target cell as an annealed duplex siRNA, or as single stranded sense and anti-sense nucleic acid sequences that once within the target cell anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

The shRNAs to be to be used in the methods of the present invention comprise a single stranded “loop” region connecting complementary inverted repeat sequences that anneal to form a double-stranded “stem” region. Structural considerations for shRNA design are discussed, for example, in McManus et al., RNA 2002; 8: 842-850. In certain embodiments the shRNA may be a portion of a larger RNA molecule, e.g., as part of a larger RNA that also contains U6 RNA sequences (Paul et al., Nature Biotechnol. 2002, 20:505-508).

In preferred embodiments the loop of the shRNA is from about 0 to about 9 nucleotides in length. In preferred embodiments the double-stranded stem of the shRNA is from about 19 to about 33 base pairs in length. In preferred embodiments, the 3′ end of the shRNA stem has a 3′ overhang. In particularly preferred embodiments, the 3′ overhang of the shRNA stem is from 1 to about 4 nucleotides in length. In preferred embodiments, shRNAs have 5′-phosphate and 3′-hydroxyl groups.

Although the RNAi molecules useful according to the invention preferably contain nucleotide sequences that are fully complementary to a portion of the target locus, 100% sequence complementarity between the RNAi probe and the target locus is not required to practice the invention.

RNA molecules useful for RNAi may be chemically synthesized, for example using appropriately protected ribonucleoside phosphoramidites and a conventional DNA/RNA synthesizer. RNAs produced by such methodologies tend to be highly pure and to anneal efficiently to form siRNA duplexes or shRNA hairpin stem-loop structures. Following chemical synthesis, single stranded RNA molecules are deprotected, annealed to form siRNAs or shRNAs, and purified (e.g., by gel electrophoresis or High Pressure Liquid Chromatography).

Alternatively, standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donzé and Picard, Nucleic Acids Res. 2002, 30:e46; and Yu et al., Proc. Natl. Acad. Sci. USA 2002, 99:6047-6052). Similarly, an efficient in vitro protocol for preparation of shRNAs using T7 RNA polymerase has been described (Yu et al., Proc. Natl. Acad. Sci. USA 2002, 99:6047-6052). The sense and antisense transcripts may be synthesized in two independent reactions and annealed later or may be synthesized simultaneously in a single reaction.

RNAi probes may be formed within a cell by transcription of RNA from an expression construct introduced into the cell. For example, a protocol and expression construct for in vivo expression of siRNAs is described in Yu et al., supra. Similarly, protocols and expression constructs for in vivo expression of shRNAs have been described (Brummelkamp et al., Science 2002, 296:550-553; Sui et al., Proc. Natl. Acad. Sci USA 2002, 99:5515-5520; Yu et al., supra; McManus et al., RNA 2002, 8:842-850; Paul et al., Nature Biotechnol. 2002, 20:505-508.

The expression constructs for in vivo production of RNAi probes comprise RNAi probe encoding sequences operably linked to elements necessary for the proper transcription of the RNAi probe encoding sequence(s), including promoter elements and transcription termination signals. Preferred promoters for use in such expression constructs include the polymerase-III HI-RNA promoter (see, e.g., Brummelkamp et al., supra) and the U6 polymerase-III promoter (see, e.g., Sui et al., supra; Paul, et al. supra; and Yu et al., supra). The RNAi probe expression constructs may further comprise vector sequences that facilitate the cloning and propagation of the expression constructs. Standard vectors useful in the current invention are well known in the art.

The RNAi probes to be used in the methods of the present invention comprise nucleic acid sequences that are complementary to the nucleic acid sequence of a portion of the target locus. In certain embodiments, the portion of the target locus to which the RNAi probe is complementary is at least about 15 nucleotides in length. In preferred embodiments, the portion of the target locus to which the RNAi probe is complementary is at least about 19 nucleotides in length. The target locus to which an RNAi probe is complementary may represent a transcribed portion of the genome (i.e., a gene) or an untranscribed portion of the genome (e.g., intergenic regions, repeat elements, etc.).

The RNAi molecules may include one or more modifications, either to the phosphate-sugar backbone or to the nucleoside. For example, the phosphodiester linkages of natural RNA may be modified to include at least one heteroatom, such as nitrogen or sulfur. In this case, for example, the phosphodiester linkage may be replaced by a phosphothioester linkage. Similarly, bases may be modified to block the activity of adenosine deaminase. Where the RNAi candidate or probe is produced synthetically, or by in vitro transcription, a modified ribonucleoside may be introduced during synthesis or transcription.

### Antisense Nucleic Acids

In a specific embodiment, to achieve inhibition of expression of sFRP genes, the nucleic acids of the invention can be used to design antisense nucleic acids. An antisense oligonucleotide is typically 18 to 25 bases in length and is designed to bind to a selected mRNA. This binding prevents translation of that specific mRNA, inhibiting production of the corresponding protein. The antisense nucleic acids of the invention comprise at least 6 nucleotides and are preferably comprised of from 6 to about 50 nucleotides. In specific aspects, the antisense oligonucleotides comprise at least 10 nucleotides, at least 15 nucleotides, at least 100 nucleotides, or at least 200 nucleotides.

Preferably, the antisense nucleic acids of the invention comprise sequences complementary to at least a portion of the corresponding mRNA. However, absolute complementarity is not required as long as formation of a stable duplex (for single stranded antisense oligonucleotides) or triplex (for double-stranded antisense oligonucleotides) can be achieved. The ability to hybridize will depend on both the degree of complementarity and the length of the antisense nucleic acid. Generally, the longer the antisense nucleic acid, the more base mismatches with the corresponding mRNA it may contain. One skilled in the art can ascertain a tolerable degree of mismatch by use of standard procedures to determine the melting point of the hybridized complex.

The antisense oligonucleotides can be DNA or RNA or chimeric mixtures or derivatives or modified versions thereof, single-stranded or double-stranded. The antisense oligonucleotides can be modified at the base moiety, sugar moiety, or phosphate backbone. The antisense oligonucleotides may include other appending groups such as peptides, or agents facilitating transport across the cell membrane (see, e.g., Letsinger et al., Proc. Natl. Acad. Sci. USA 1989, 86:6553-6556; Lemaitre et al., Proc. Natl. Acad. Sci. USA 1987, 84:648-652; PCT Publication No. WO 88/09810) or blood-brain barrier (see, e.g., PCT Publication No. WO 89/10134), hybridization-triggered cleavage agents (see, e.g., Krol et al., BioTechniques 1988, 6:958-976), intercalating agents (see, e.g., Zon, Pharm. Res. 1988, 5:539-549), etc. In another embodiment, the antisense oligonucleotides can be α-anomeric oligonucleotides. An α-anomeric oligonucleotide forms specific double-stranded hybrids with complementary RNA in which, contrary to the usual β-units, the strands run parallel to each other (Gautier et al., Nucl. Acids Res. 1987; 15:6625-6641). In yet another embodiment, the antisense oligonucleotides can be morpholino antisense oligonucleotides (i.e., oligonucleotides in which the bases are linked to 6-membered morpholine rings, which are connected to other morpholine-linked bases via non-ionic phosphorodiamidate intersubunit linkages). Morpholino oligonucleotides are resistant to nucleases and act by sterically blocking transcription of the target mRNA.

Similarly to the above-described RNAi molecules, the antisense oligonucleotides of the invention may be synthesized by standard methods known in the art, e.g., by use of an automated synthesizer. Antisense nucleic acid of the invention can be also produced intracellularly by transcription from an exogenous sequence. For example, a vector can be introduced in vivo such that it is taken up by a cell, within which cell the vector or a portion thereof is transcribed, producing an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. In another embodiment, “naked” antisense nucleic acids can be delivered to adherent cells via “scrape delivery.” This method involves scraping the cells from the culture plate; and transferring the scraped cells to another plate where they are allowed to re-adhere. Scraping the cells from the plate pulls adhesion plaques from the cell membrane, generating small holes which allow antisense oligonucleotides to directly enter the cytosol for up to one minute, by which time the holes have resealed.

### Ribozyme Inhibition

In another embodiment, the level of expression of sFRPs of the present invention may be inhibited by using sFRP nucleic acids to design ribozymes useful to decrease the level of gene expression.

Ribozyme molecules designed to catalytically cleave mRNA transcripts can be used to prevent translation of mRNA and, therefore, expression of the gene product. Ribozymes are enzymatic RNA molecules capable of catalyzing the sequence-specific cleavage of RNA (for a review, see Rossi, Current Biology 1994, 4:469-471). The mechanism of ribozyme action involves sequence-specific hybridization of the ribozyme molecule to complementary target RNA, followed by an endonucleolytic cleavage event. The composition of ribozyme molecules must include (i) one or more sequences complementary to the target gene mRNA and (ii) a catalytic sequence responsible for mRNA cleavage (see, e.g., U.S. Pat. No. 5,093,246).

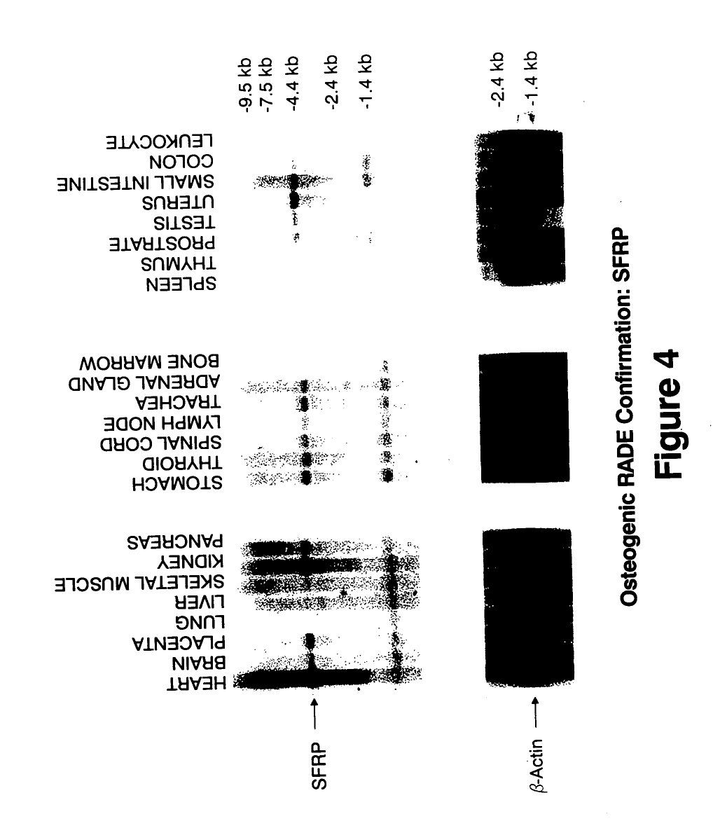

According to the present invention, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Myers, Molecular Biology and Biotechnology: A Comprehensive Desk Reference, VCH Publishers, New York, 1995 (see especially FIG. 4, page 833) and in Haseloff and Gerlach, Nature 1988, 334:585-591.

Preferably the ribozymes of the present invention are engineered so that the cleavage recognition site is located near the 5′ end of the corresponding mRNA, i.e., to increase efficiency and minimize the intracellular accumulation of non-functional mRNA transcripts.

As in the case of RNAi and antisense nucleic acids, the ribozymes of the invention can be composed of modified oligonucleotides (e.g., for improved stability, targeting, etc.) and should be delivered to cells that express the target genes in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the protein and inhibit translation. Because ribozymes, unlike antisense molecules, are catalytic, a lower intracellular concentration is required for efficacy.

Ribozymes of the invention may be prepared by any method known in the art for the synthesis of DNA and RNA molecules, as discussed above. Ribozyme technology is described further in Intracellular Ribozyme Applications: Principals and Protocols, Rossi and Couture eds., Horizon Scientific Press, 1999.

### Industrial Applicability

The compositions described above provide the components for an assay to screen for agents and pharmaceutical compounds that are agonists or antagonists of a Wnt receptor in a suitable cell.

It is also anticipated that the sFRP polynucleotides of the invention will have utility as diagnostic agents or detecting genetic abnormalities associated with genes encoding sFRP or with one or more genes involved in the Wnt signaling pathway. Such genetic abnormalities include point mutations, deletions, or insertions of nucleotides. Any of several genetic screening procedures may be adapted for use with probes enabled by the present invention, including restriction fragment length polymorphism (RFLP) analysis, ligase chain reaction, or PCR. Mutations in this gene indicate increased risk of developmental abnormalities.

As provided in more detail below, the proteins and fragments thereof are useful in a cell-free and cellular in vitro assay system to screen for agents and pharmaceutical compounds which either inhibit or augment the Wnt-receptor pathway and apoptosis and to test possible therapies for disorders associated with this pathway, e.g., bone formation diseases, carcinogenesis, and cardiovascular diseases. Embryogenesis also can be modulated.

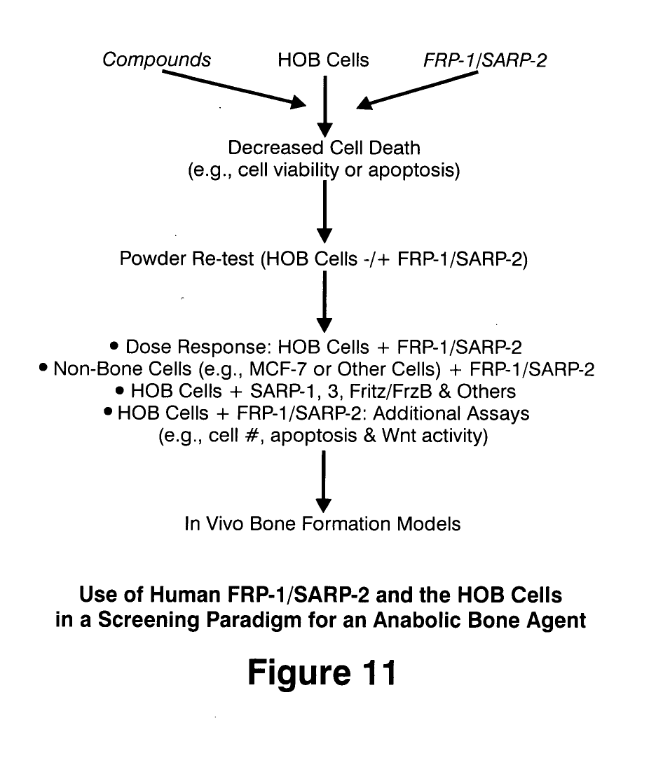

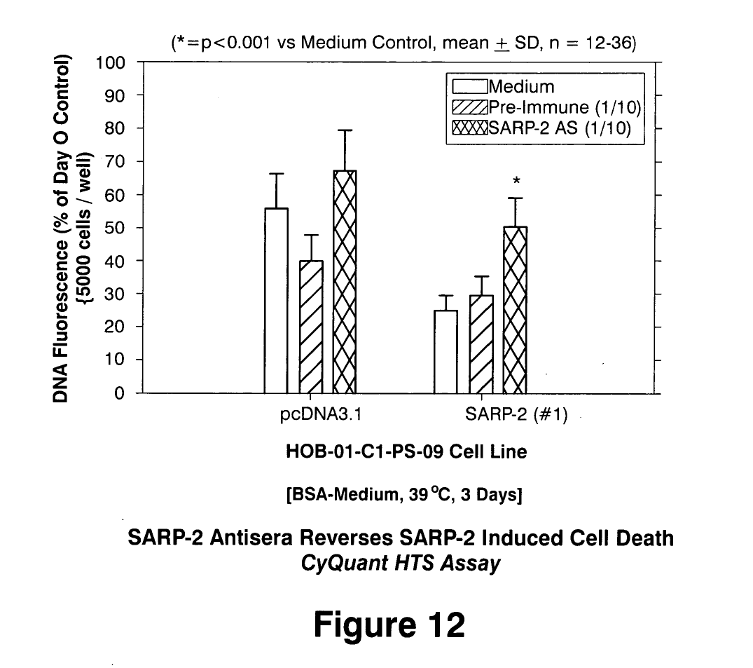

In vitro and in vivo drug screening assays can be used to identify activators or inhibitors of the sFRP protein. For example, activators or agonist, or alternatively inhibitors or antagonists, of sFRP-1 can be screened for by comparing the activity of a test compound in an sFRP-1 +/+mouse with that in an sFRP −/− mouse. Increase or decrease in sFRP-1 specific-activity will be indicated by a phenotypic change in the sFRP-1 +/+ mouse relative to the sFRP-1 −/− mouse. For example, an increase in cartilage growth in an sFRP-1 +/+ animal as compared to an sFRP-1 −/− animal in the presence of a drug may indicate activation of sFRP, while a decrease may indicate inhibition of the sFRP activity. FIGS. 11 and 12 are illustrative of in vitro assays that can be performed in order to identify modulators of sFRP activity. Reporter gene based assays are also provided by the invention. For example, an agonist screen involves the detection of a decrease in reporter gene (e.g. green fluorescent protein, β-galactosidase) expression by a host cell contacted with a test compound.

A variety of compounds may be screened using methods of the present invention. They include peptides, macromolecules, small molecules, chemicals and biological mixtures. Such compounds may be biological, synthetic, organic, or inorganic compounds.

In the present invention suitable cells are used for preparing diagnostic assays, for the expression of sFRPs or for preparing nucleotide-based diagnostic kits. The cells may be made or derived from yeast, bacteria, fungi, or viruses. In preferred embodiments, the cells are hOB cells, in particular a novel immortalized pre-osteocytic cell line referred to as hOB-01-C1-PS-09 cells (which are deposited with American Type Culture Collection in Manassas, Va. with the designation PTA-785), and osteoblast cells having the identifying characteristics of hOB-01-C1-PS-09 cells as well as osteoblast cells made therefrom, e.g. progeny. Immortalized refers to a substantially continuous and permanently established cell culture with substantially unlimited cell division potential. That is, the cells can be cultured substantially indefinitely, i.e., for at least about 6 months under rapid conditions of growth, preferably much longer under slower growth conditions, and can be propagated rapidly and continually using routine cell culture techniques. Alternatively stated, the cells of the present invention can be cultured for at least about 100, 150 or 200 population doublings. These cells produce a complement of proteins characteristic of normal human osteoblastic cells and are capable of osteoblastic differentiation. They can be used in cell culture studies of osteoblastic cell sensitivity to various agents, such as hormones, cytokines, and growth factors, or in tissue therapy. These cells are a post-senescent subclone of hOB-01-C1 cell line, as previously disclosed by Bodine et al., Endocrinology 1996, 137:4592-4604.

Since, as we report herein, sFRPs are new drug targets for osteoporosis, certain embodiments relate to the expression of genes or nucleic acids that encode all or portion of at least one sFRP protein. Expression of such nucleic acids or genes in human osteoblast (hOB) cell lines correlates with accelerated cell death and apoptosis. The hOB-01C1PS-09 cells of this invention are particularly useful over other hOB cells since the present “-09” cells are adult osteoblast cells. In addition, these cells are osteocytic (i.e., mature cells) in comparison to other hOB cells that are often osteoblastic. Furthermore, the hOB-01-C1-PS-09 cells express very low levels of FRP-1/SARP-2 message. Consequently, the hOB-01-C1-PS-09 cell line is a unique in vitro model to study the effects of FRP-1/SARP-2 reintroduction and over-expression. Another important feature of the hOB-01-C1-PS-09 cells is that they can be used for both transient and stable transfection studies. Some of the many advantages of this cell over the parental hOB-01-C1 cells are as follows: the hOB-01-C1-PS-09 cells are truly immortal, they divide 2- to 3-times faster at 34° C., and yet they retain many of the pre-osteocytic characteristics of the parental cells.

The hOB-01-C1-PS-09 cells will be useful for establishing stable cell lines that over-express potential osteoporotic drug targets. Such stable cell lines will then be valuable for characterizing these drug targets, as well as for developing high throughput screens and assays to identify compounds that regulate them.

Agents according to the present invention may be identified by screening in high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time (see, e.g., U.S. Pat. Nos. 5,585,277, 5,679,582, and 6,020,141).

### EXAMPLES

The present invention is further described by the following examples. The examples are provided solely to illustrate the invention by reference to specific embodiments. These exemplifications, while illustrating certain specific aspects of the invention do not portray the limitations or circumscribe the scope of the invention.

### Example 1

### Generation and Analysis of hOB Cells

The hOB-01-C1 cells are a conditionally-transformed cell line derived from adult human bone that faithfully exhibit a pre-osteocytic phenotype. These cells were transformed with a temperature-sensitive large T-antigen (tsA 209) and proliferate at the permissive temperature of 34° C. when the T-antigen mutant is active; however, the cells stop dividing at the non-permissive temperature (> or =37° C.) when the T-antigen mutant is inactive. Although the hOB-01-C1 cells are the first osteocyte cell line to be established and are suitable for exploratory research, they have some disadvantages for drug discovery. Like other SV-40 large T-antigen transformed human cell lines, the hOB-01-C1 cells undergo crisis and senesce after 15-20 passages in culture. Thus, although often referred to as “immortal”, such cell lines are actually only “extended-life”. The hOB-01-C1 cells also proliferate slowly in culture at 34° C. with a doubling time of about once every 5 to 6 days. In order to overcome some of these draw-backs, the hOB-01-C1-PS-09 cell line was established.

hOB-01-C1-PS-09 cells were developed by passaging the parental hOB-01-C1 cell line beyond the crisis point (i.e., passages 15-20) until proliferation resumed (passages 20-25). The post-senescent cells were then expanded in culture and sub-cloned. Clones were characterized using reverse transcriptase-polymerase chain reaction (RT-PCR) analysis to measure the levels of expression of parathyroid hormone (PTH)-1 receptor mRNA. The hOB-01-C1-PS-09 cells were chosen for further characterization, since this clone expressed the highest level of PTH-1 receptor message at 39° C. This cell line went through approximately 20-25 population doublings during the cloning and expansion procedure, and has subsequently been passaged over 50-times. These cell line can be passaged hundreds of times. Thus, the hOB-01-C1-PS-09 cells are truly an immortal cell line.

Like the parental hOB-01-C1 cell line, the hOB-01-C1-PS-09 cells fail to form monolayer cultures and leave spaces on the tissue culture dishes. The cells also appear to form cell-to-cell contacts through long cellular processes. From analysis by electron microscopy, the cells possess finger-like cellular projections that are reminiscent of pre-osteocytes, and these processes form gap junctions when they contact adjacent cells. As with the parental cell line, the hOB-01-C1-PS-09 cells also express the tsA 209 large T-antigen as determined by immunocytochemistry.

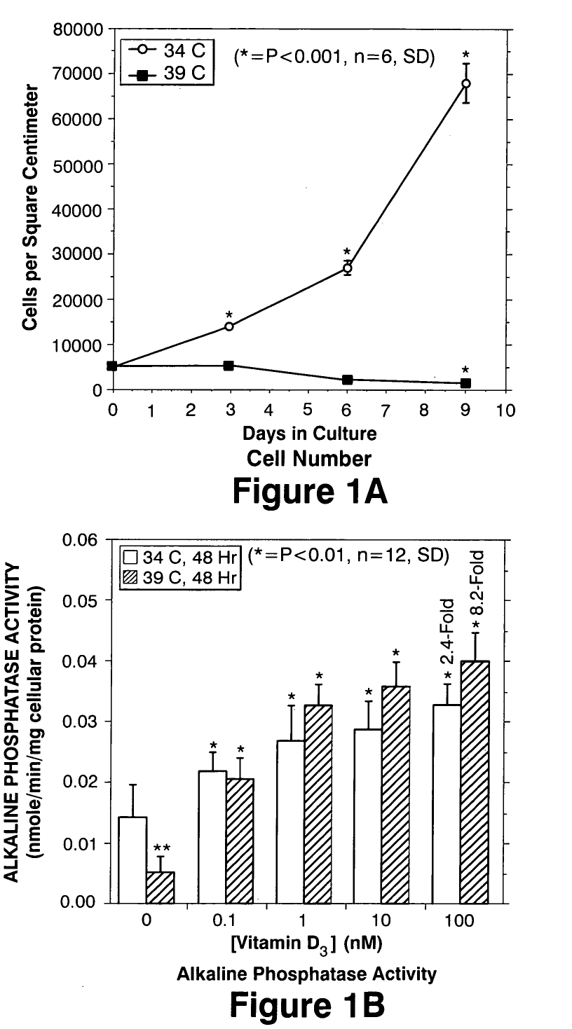

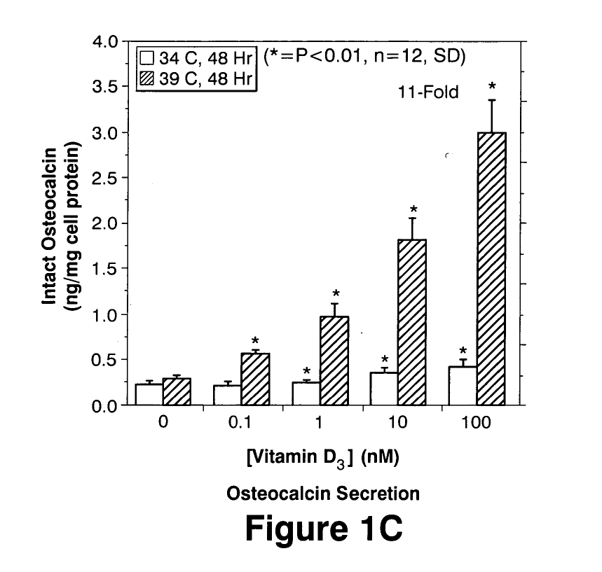

Unlike the parental hOB-01-C1 cell line, the hOB-01-C1-PS-09 cells proliferate 2- to 3-times faster at the permissive temperature with a doubling time of once every 2-3 days (FIG. 1). However, like the parental cells, the hOB-01-C1-PS-09 cells stop dividing at the non-permissive temperature (FIG. 1A). This observation indicates that although the cells have passed through the crisis point and become immortal, they still require an active T-antigen for proliferation. Moreover, as with the parental cell line, the hOB-01-C1-PS-09 cells require inactivation of the T-antigen in order to exhibit an enhanced osteocytic phenotype. Alkaline phosphatase and osteocalcin are two important markers of the osteocytic lineage. As shown in FIG. 1B, the ability of vitamin D3-treatment to up-regulate alkaline phosphatase activity is enhanced about 4-fold when the cells are incubated at 39° C. Furthermore, as depicted in FIG. 1C, vitamin D3 is unable to induce osteocalcin secretion from the cells at 34° C.

However, the secosteroid up-regulates production of this bone-specific matrix protein 11-fold when the cells are incubated at 39° C. It should be noted that the basal levels of alkaline phosphatase expression and osteocalcin secretion by the hOB-01-C1-PS-09 cells are similar to the parental cell line. Thus, both morphologically and biochemically, the hOB-01-C1-PS-09 cells resemble the parental hOB-01-C1 cells and are therefore a reliable in vitro model to study human pre-osteocyte biology.

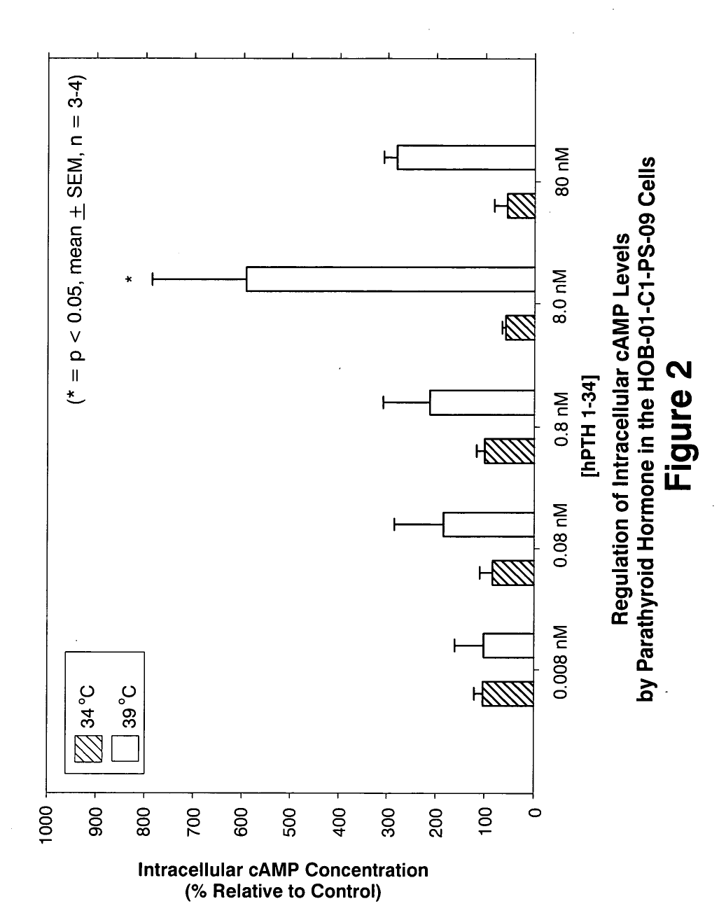

As mentioned above, the hOB-01-C1-PS-09 cell line was selected for further characterization based on its high-level of PTH-1 receptor mRNA expression. Incubation of the cells for 48 hr at 39° C. increased the steady-state message levels for the PTH-1 receptor by 7-fold when compared to cells maintained at 34° C. Because PTH-1 receptor expression is a marker of osteoblast/osteocyte differentiation, this is another indication that the cells exhibit a more pronounced osteocytic phenotype at the non-permissive temperature. Consistent with this enhanced PTH-1 receptor expression, preincubation of the hOB-01-C1-PS-09 cells for 48 hr at 39° C. followed by treatment with increasing concentrations of human PTH 1-34 (hPTH 1-34) for 10 min at 37° C. generates a dose-dependent 5- to 6-fold increase in intracellular cyclic-adenosine monophosphate (cAMP) levels (FIG. 2). In contrast, preincubation of the cells at 34° C. does not result in a subsequent increase in cAMP concentrations after hPTH 1-34 treatment. Thus, both PTH-1 receptor expression and responsiveness are enhanced following inactivation of the tsA-209 T-antigen. One potential utility for this cAMP assay would be the ability to characterize the activities of PTH-analogs or -mimetics in an important target cell under conditions where levels of PTH-1 receptor expression are dramatically altered.

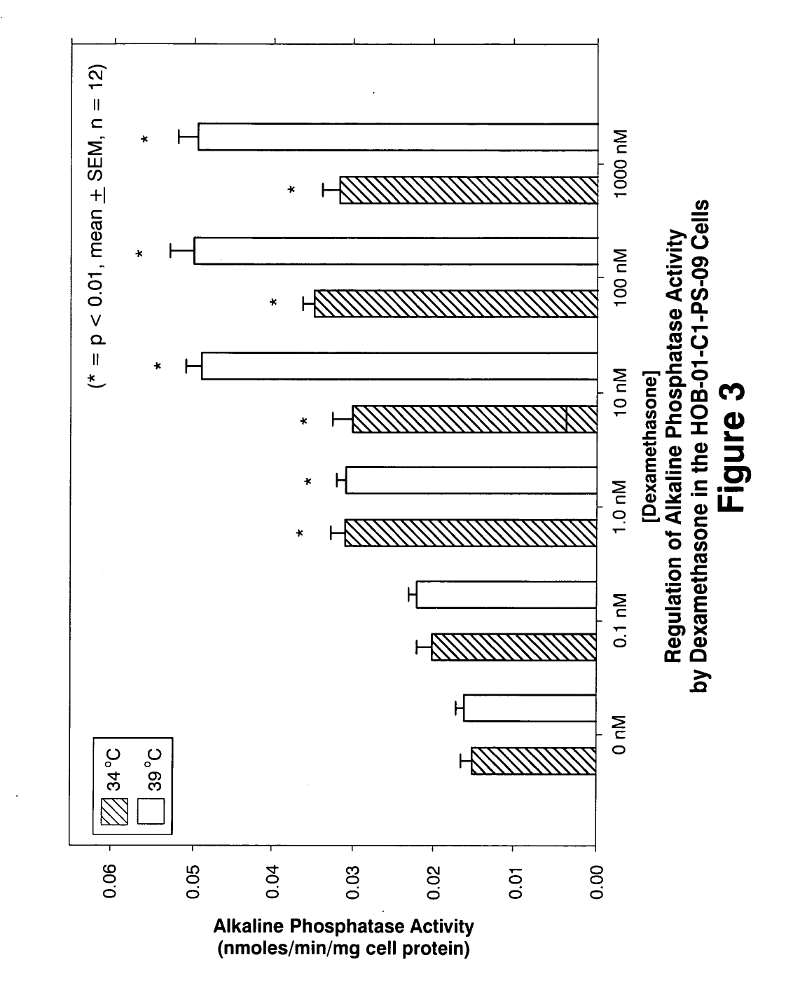

In addition to vitamin D3 and PTH, the hOB-01-C1-PS-09 cells also respond to additional bone-active agents: for example, glucocorticoids and transforming growth factor (TGF)-β1. Treatment of the cells with the synthetic glucocorticoid, dexamethasone, up-regulates alkaline phosphatase activity approximately 2-fold at 34° C. (FIG. 3), and this effect is once again enhanced when the cells are incubated at 39° C. Likewise, treatment of the cells with recombinant human (rh) TGF-β1 at 39° C. results in a dose-dependent decrease in hepatocyte growth factor (HGF) secretion. HGF has been shown to act as a chemotactic factor for osteoclasts, and may therefore play a role in regulating bone resorption.

An important property of osteocytes is the ability to respond to mechanosensory stimulation, such as that which occurs during weight-bearing exercise. One method to simulate this stimulatory effect in vitro is through the use of a Flexercell Strain Unit (Flexcell International, Hillsborough, N.C.). hOB-01-C1-PS-09 cells were seeded onto BioFlex type I collagen coated 6-well tissue culture dishes and incubated at 34° C. for 24 hr. The cells were then pre-incubated in serum-free medium at either 34° C. or 39° C. for an additional 24 hr, and then subjected to a physiologically-relevant strain (3400 HE, 2 Hz, 7200 cycles) at 37° C. using an FX-3000 Flexercell Strain Unit. After the strain-treatment, the cells were incubated for 4.5 hr, at which time the conditioned medium was collected and analyzed for the presence of nitric oxide (NO). It has been previously reported that in vitro mechanosensory stimulation or shear-stress of rodent and chick osteoblasts and osteocytes stimulates NO production. Mechanosensory stimulation (i.e., “Flex”) of the hOB-01-C1-PS-09 cells enhanced the production of NO by 10- to 18-fold. Consequently, these data suggest that this cell line will be a useful in vitro model to study the molecular mechanisms of mechanosensory stimulation.

Additional experiments establish that the hOB-01-C1-PS-09 cells can be used for both transient and stable transfection studies. This cell line can be transfected using the Tfx-20 lipofection reagent (Promega, Madison, Wis.). In such an experiment, the cells are seeded into 24-well tissue culture dishes at varying densities and then transfected with 0.25 μg/well of β-galactosidase and luciferase expression plasmids (total DNA=0.5 μg/well). After a 48 hr incubation at either 34° C. or 39° C., cell lysates are assayed for β-galactosidase and luciferase activity. From such experiments, results establish that the levels of either β-galactosidase or luciferase expression increase with increasing cell number. Moreover, when the luciferase expression is normalized to β-galactosidase expression in order to control for transfection efficiency, the level of luciferase expression is 2- to 3-fold higher when the cells are incubated at 34° C. Since luciferase expression is under the control of the SV-40 promoter, this observation is consistent with the tsA 209 T-antigen being inactivated at 39° C. Consequently, since these cells are both immortal and capable of being transfected, they can be used to develop stable over-expressing cell lines in a human pre-osteocytic background.

### Example 2

### Isolation of sFRP

The hOB sFRP gene fragment was identified using RADE (rapid analysis of differential expression) technology as described by Shiue 1997 Drug Develop. Res. 41: 142-159, the whole of which is incorporated herein. Three hOB cell lines (hOB-03-C5, hOB-03-CE6 and hOB-01-C1), representing three distinct stages of differentiation (proliferative, mature and preosteocytic, respectively) were used to isolate and identify sFRP. The hOB cell lines were established and cultured as previously described (Bodine et al., J. Bone Miner. Res. 1996, 11:806-819; Bodine et al., Endocrinology 1996, 137:4592-4604; Bodine et al., J. Cell. Biochem. 1997, 65:368-387). These cell lines were immortalized with a temperature-sensitive simian virus (SV) 40 large T-antigen and exhibited a transformed phenotype at the permissive temperature (34° C.) when the T-antigen mutant was active. However, in contrast to osteosarcoma cells (Stein and Lian, Endocrine Rev. 1993, 14:424-442), the hOB cell lines were faithful to the proliferation/differentiation relationship at the nonpermissive temperature (>37° C.) when the T-antigen mutant is inactivated. The cell lines were seeded into 150 mm dishes at −40,000 cells/cm2 with growth medium (D-MEM/F-12 containing 10% (v/v) heat-inactivated fetal bovine serum (FBS), 1% (v/v) Penicillin-Streptomycin and 2 mM GlutaMAX-1) and incubated overnight at 34° C. The next day, the medium was removed, the cells were rinsed with phosphate-buffered saline (PBS), 20 ml of serum-free medium was added to the dishes (phenol red-free D-MEM/F-12 (Gibco/BRL) containing 0.25% (w/v) bovine serum albumin (BSA, Pentex crystallized, Bayer), 1% (v/v) Penicillin-Streptomycin, 2 mM GlutaMAX-1, 50 mM ascorbate-2-phosphate (Wako), and 10 nM menadione sodium bisulfite (vitamin K₃)), and the dishes were incubated at 39° C. for 24 hr. The next day, the medium was removed and the cells were treated at 39° C. for an additional 24 hr with 20 ml of fresh serum-free medium containing either vehicle (Control), 8 nM human parathyroid hormone 1-34 (PTH), 100 nM prostaglandin E2 (PGE2) or 0.1 nM human transforming growth factor-β1 (TGF-01). PTH, PGE2 and TGF-01 are known osteogenic agents (Whitfield and Morley, Trends Pharmaceut. Sci. 1995, 16:382-386; Jee and Ma, Bone 1997, 21:297-304; Centrella et al., Endocrine Rev. 1994, 15:27-39). After the treatment period, the dishes were rinsed with PBS and total cellular RNA was isolated from the nontreated and treated cells using Trizol according to the manufacturer's instructions (Gibco Invitrogen). RADE was then performed with the isolated RNA samples as above; the regulated gene fragments were identified, cloned and sequenced. These experiments identified a total of 82 differentially expressed genes. A BLAST (basic local alignment search tool) search of the public data bases was performed on the RADE-obtained gene fragments; one of the gene fragments was highly homologous to mouse sFRP-1. This gene fragment was identified during RADE using the following primer pair: 5′-AAGCTTTTTTTTTTTA-3′ (HT11A)₃′ end (reverse primer) and 5′-AAGCTTGATTGCC-3′ (H-AP1) 5′ end (forward primer), which sequences are SEQ ID NO: 3 and SEQ ID NO: 4, respectively. The expression and regulation of the gene fragment having homology to mouse cDNA sFRP-1 was confirmed by Northern blot analysis.

### Example 3

### Characterization of hOB sFRP

The results from RADE demonstrated that the hOB sFRP gene fragment was strongly up-regulated by PGE2 treatment in the proliferative-stage (hOB-03-C5) and maturation-stage (hOB-03-CE6) hOB cell lines, but down-regulated by TGF-β1 treatment in the pre-osteocytic (hOB-01-C1) cell line. Moreover, basal expression of this gene was dramatically increased in the pre-osteocytic cells (hOB-01-C1), suggesting that hOB sFRP gene expression is linked to the osteoblast differentiation process.

### Example 4

### Sequence Analysis of the hOB sFRP Gene Fragment

The hOB sFRP gene fragment identified in Example 2, above, containing 276 base pair (bp) was cloned, sequenced, and a further subjected to a BLAST search of the public databases. This search revealed that this gene was homologous to two other previously identified cDNAs. Sequence alignment indicated that the hOB sFRP gene fragment shared 77% sequence identity to the 3′-end of mouse sFRP-1 gene (GenBank™ Accession #U88566; Rattner et al. Proc. Natl. Acad. Sci. USA 1997, 94:2859-2863). The hOB sFRP gene fragment also exhibited significant homology (87%) to the 3′-end of a related bovine cDNA called frizzled-related protein A (FrzA, GenBank™ Accession #U85945). Additionally, the hOB sFRP gene fragment was very homologous to at least three expressed sequence tags (ESTs), human clone TM010 (GenBank™ Accession #U54715), human CA11 tumor suppresser (GenBank™ Accession #U69122) and a human infant brain EST (GenBank™ Accession #H16753, H16861).

### Example 5

### Regulation of the hOB sFRP by Osteogenic Agents

In order to confirm the regulation of hOB sFRP gene expression by different osteogenic agents (i.e., the DNA fragment identified in Example 2, above), the hOB cell lines were treated with PTH, PGE2, and TGF-β1; RNA was then isolated for Northern hybridizations. The experiments were performed as follows. The hOB cell lines were seeded into 150 mm dishes and treated as described in Example 2, except that polyA+ RNA was isolated from total cellular RNA using Oligotex mRNA maxi kits as described by the manufacturer (Qiagen, Valencia Calif.). Northern blot analysis was performed using either the excised RADE hOB sFRP gene fragment, the cloned hOB sFRP gene fragment or the cloned full-length hOB sFRP cDNA as a 32P-labeled probe (as described in Bodine et al., J. Bone Miner. Res. 1996, 11:806-819, which is incorporated herein). Each of these probes detected a 4.4-4.6 Kb message in the hOB cells. Expression of the hOB sFRP mRNA was normalized to either glyceraldehyde phosphate dehydrogenase (GAPDH) mRNA or β-actin mRNA using the corresponding 32P-DNA probes. Treatment of the proliferative-stage hOB-03-C5 cells with 100 nM PGE2 for 24 hr completely up-regulated the expression of a ˜4.6 kilobase pair (Kb) message, confirming the regulation of this gene by PGE2. When the cloned hOB SRFP gene fragment was used as a probe, a predominant mRNA of ˜4.4 Kb was observed in cells treated with PGE2, confirming that this message is indeed the sFRP gene. This mRNA corresponds in size to the transcript for the human FRP-1/SARP-2 gene (Finch et al., Proc. Natl. Acad. Sci. USA 1997, 94:6770-6775; Melkonyan et al., Proc. Natl. Acad. Sci. USA 1997, 94:13636-13641). Northern blot analysis also confirmed the up-regulation of hOB sFRP mRNA expression in maturation-stage hOB-03-CE6 cells treated with PGE2. In addition, basal expression of this gene was elevated in pre-osteocytic hOB-01-C1 cells. This basal level expression was suppressed by 35% following treatment with 8 nM PTH. Furthermore, PGE2 treatment of the maturation-stage hOB-03-CE6 cells elevated sFRP expression to the level that was expressed basely by the pre-osteocytic cells, implying that up-regulation of sFRP by PGE2 in the osteoblastic cells is related to the enhancement of cellular differentiation. Lastly, treatment of the pre-osteocytic hOB-01-C1 cells with 100 pM TGF-β1 for 24 hr suppressed hOB sFRP mRNA levels by 80%.

To confirm that hOB sFRP message levels change with increasing cellular differentiation, total RNA was isolated from the pre-osteoblastic hOB-03-C5 cells, the mature osteblastic hOB-03-CE6 cells, the pre-osteocytic hOB-01-C1 cells and the mature osteocytic hOB-05-T1 cells. Basal sFRP mRNA levels were then measured by TaqMan quantitative RT-PCR. When compared to the hOB-03-C5 cells, basal sFRP message levels increased about 4-fold in the hOB-03-CE6 cells and about 23-fold in the hOB-01-C1 cells. On the other hand, sFRP mRNA levels declined to about 0.5-fold in the hOB-05-T1 cells. Thus, of the cells in the osteoblast lineage, the pre-osteocyte appears to express the highest levels of hOB sFRP message.

### Example 6

### Kinetics of hOB sFRP Expression

Proliferative hOB-03-C5 cells were seeded into 150 mm dishes and treated with increasing concentrations of PGE₂ for 24 hr or with 100 nM PGE₂ for varying lengths of time as described in Example 2. PolyA+ RNA Northern blot analysis was performed with the excised RADE hOB sFRP 276 bp gene fragment or a cloned 1.1 Kb hOB SRFP gene fragment as a ³²P-labeled probe. Treatment of the proliferative-stage hOB-03-C5 cells with increasing concentrations of PGE₂ up-regulated hOB sFRP mRNA expression in a dose-dependent manner with an EC₅₀ of approximately 8 nM. Likewise, treatment of the mature hOB-03-CE6 cells with increasing concentrations of PGE₂ also up-regulated hOB sFRP mRNA levels in a dose-dependent manner, although the EC₅₀ of PGE₂ for this response was about 10-times higher than in the hOB-03-C5 cells.

Treatment of the hOB-03-C5 cells with 100 nM PGE2 for 2 to 24 hr up-regulated hOB sFRP mRNA expression in a time-dependent manner. A significant increase in sFRP gene expression was observed after 2 to 4 hr of treatment, and the steady-state mRNA levels continued to increase up to 24 hr after the addition of PGE2 to the cell culture medium. These results suggest that sFRP may be a late-response gene to PGE2 treatment of the hOB cells, and implies that it may be under the secondary control of another gene product. Similar results were also obtained with the mature hOB-03-CE6 cells, although the fold-increase in expression of the hOB sFRP message was not as great as it was in the hOB-03-C5.

In a similar manner, the hOB-01-C1 cells were seeded into 100 mm dishes and treated for 24 hr with increasing concentrations of TGF-β1. Total RNA was then isolated from the cells, and hOB sFRP mRNA was measured by TaqMan quantitative RT-PCR. TGF-01 suppressed sFRP gene expression by approximately 70% in a dose-dependent manner with an IC₅₀ of about 4 pM.

In addition to PGE2, treatment of either the hOB-03-C5 cells or the hOB-03-CE6 cells with interleukin (IL)-1β, 9-cis-retinoic acid or all-trans-retinoic acid also increases hOB sFRP mRNA levels. On the other hand, as with TGF-β1, treatment of various hOB cell lines with PTH, bone morphogenetic protein (BMP)-2, insulin-like growth factor (IGF)-I, 17β-estradiol (17β-E2), vitamin D3 (VD3), dexamethasone (Dex), or fetal bovine serum (FBS) suppresses hOB sFRP message expression. Overall, there is a good correlation between the abilities of these various agents to either increase or decrease hOB sFRP expression, and their ability to either enhance or suppress osteoblast/osteocyte apoptosis (see for example Manolagas, Endocrine Reviews 2000, 21:115-137).

### Example 7

### Tissue Distribution of hOB sFRP

In order to determine the tissue distribution for the expression of the hOB sFRP gene, multiple human tissue poly(A)+ RNA Northern blots (obtained from Clontech) were probed with the excised RADE hOB sFRP gene fragment as described in Example 5. When the RADE fragment was used to probe the poly (A)+ RNA Northern blots, a ˜4.4 Kb transcript was expressed by several tissues (FIG. 4). When these results were normalized to β-actin, sFRP expression was ranked as follows:

Kidney>heart>placenta>liver=skeletal muscle=stomach=thyroid gland>adrenal gland=testis=uterus=small intestine=pancreas=brain>trachea=spinal cord=prostate=colon>spleen>lung=lymph node=bone marrow;

No expression was observed in thymus and peripheral blood lymphocytes. This expression pattern is similar to the human FRP-1/SARP-2 gene (Finch et al., Proc. Natl. Acad. Sci. USA 1997, 94:6770-6775; Melkonyan et al., Proc. Natl. Acad. Sci. USA 1997, 94:13636-13641).

### Example 8

### Distribution of sFRP in Osteoblast Cell Lines

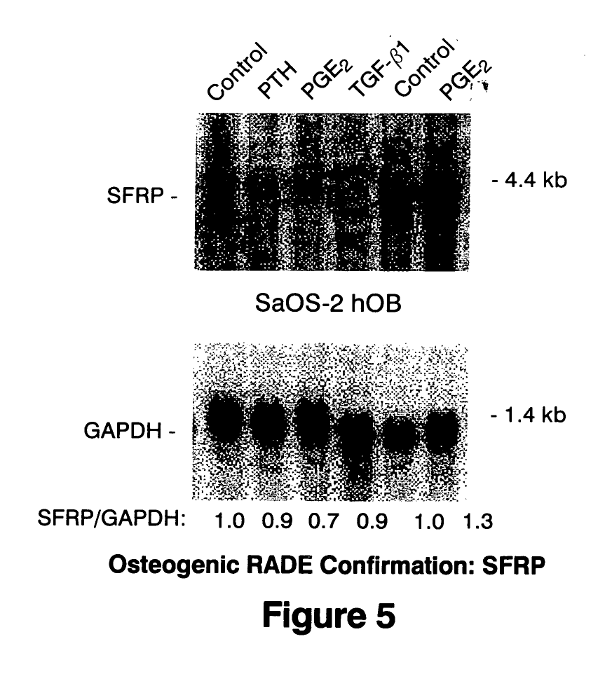

In addition to the hOB cell lines from which sFRP was initially identified, additional in vitro human osteoblast models were examined for the presence of the gene. SaOS-2 human osteosarcoma osteoblast-like cells were obtained from the American Type-Culture Collection (ATCC) and were cultured at 37° C. in McCoy's 5A Modified medium containing 10% FBS, 1% (v/v) Penicillin-Streptomycin and 2 mM GlutaMAX-1. Likewise, explant cultures of normal human osteoblasts (hOBs) were established from cancellous bone chips as previously described (Bodine et al., J. Bone Miner. Res. 1996, 11:806-819, which is incorporated herein by reference). The cells were then seeded into 150 mm dishes and treated as described in Examples 2 and 5, except that the cells were incubated at 37° C. instead of 39° C. PolyA+ RNA Northern blot analysis was performed using the cloned 1.1 Kb hOB SRFP gene fragment as a ³²P-label. SaOS-2 cells expressed relatively low basal levels of sFRP mRNA which was not regulated by treatment with either PTH, PGE₂ or TGF-β1 (FIG. 5). It was difficult to quantify expression of this gene in these cells, since the level of expression was low. In contrast, normal hOB cells expressed higher basal levels of sFRP message, and treatment of the cells with 100 nM PGE₂ for 24 hr appeared to slightly up-regulate the steady-state levels of this mRNA (˜1.3-fold). TaqMan quantitative RT-PCR analysis of these RNA samples indicated that PGE₂ upregulated sFRP 10-fold in the hOB cells. Due to the low basal level expression in osteosarcoma cells, these cells may not be satisfactory in vitro models to study the regulation of the sFRP gene. Since the gene is expressed and regulated by PGE₂ in cultures of normal human osteoblasts, the use of the hOB cell lines described in the present invention is validated for in vitro osteoblast models. Northern blot analysis and RT-PCR of total RNA isolated from a human giant cell tumor of bone failed to detect expression of the hOB sFRP mRNA in this tissue. These results suggest that osteoclast-like cells may not express this gene.

### Example 9

### Isolation of Full Length hOB sFRP cDNA

Since the cloned hOB sFRP gene fragment from RADE was identical to several human ESTs, an analysis of the EST database was performed in order to assemble the full-length cDNA for the hOB gene. This analysis suggested that the hOB sFRP was in fact the known human gene, FRP-1 (also called the secreted apoptosis-related protein-2 or SARP-2). Based on this analysis, and the observation that the mouse sFRP-1 gene is apparently homologous to the human FRP-1 gene and the human SARP-2 gene (Rattner et al., Proc. Natl. Acad. Sci. USA 1997, 94:2859-2863; Finch et al., Proc. Natl. Acad. Sci. USA 1997, 94:6770-6775; Melkonyan et al., Proc. Natl. Acad. Sci. USA 1997, 94:13636-13641), an RT-PCR-based strategy was designed to obtain the full-length 1.1 Kb hOB sFRP cDNA from both human placenta RNA and PGE₂-treated hOB-03-CE6 cell RNA. RT-PCR was performed using 1 μg of total RNA, primers that spanned the coding region of hFRP-1/SARP-2 (forward primer: 5′-GCTGGGGACTGCGCCTTTTGT-3′ SEQ ID NO: 5; reverse primer: 5′-CCTGCCCCCGGGAGAATCACTTA-3′ SEQ ID NO: 6), 35 cycles of PCR, and the Advantage-GC PCR kit (Clontech) according to the manufacturer's instructions. In order to detect expression of the mRNA, a Southern blot analysis was performed with the RT-PCR products using a ³²P-oligonucleotide probe which specifically hybridized to bases 501 to 530 of the hFRP-1/hSARP-2 coding region (refer to Bodine et al., J. Cell. Biochem. 1997, 65:368-387 for experimental details concerning RT-PCR and Southern hybridizations). The results confirmed that placenta expresses FRP-1/SARP-2 mRNA. Likewise, full-length 1.1 Kb cDNA for the hOB sFRP was isolated and was up-regulated by 100 nM PGE₂-treatment of the hOB-03-CE6 cells for 24 hr. Likewise, RT-PCR of total RNA isolated from hOB-03-C5 cells treated with PGE₂ identified a 2.2 Kb cDNA which spanned from the 5′-region of the hFRP-1/SARP-2 cDNA to the 276 bp RADE fragment at the 3′-end. These cDNA fragments were cloned into either the pcDNA3.1 (Invitrogen) mammalian expression vector (1.1 Kb cDNA) or the TA (Invitrogen) cloning vector (2.2 Kb cDNA) and sequenced. Sequence analysis of the hOB sFRP 1.1 Kb (SEQ ID NO: 1) and 2.2 Kb cDNAs enabled the assembly of a 2.6 Kb cDNA which included the transcription start site at the 5′-end and the RADE fragment at the 3′-end. A BLAST search of the public databases using the 1.1 Kb cDNA indicated that it essentially was identical to human FRP-1/SARP-2. The deduced amino acid sequence of the coding region of the sFRP cDNA is shown in SEQ ID NO: 2. SEQ ID NO: 2 contains one amino acid different from the published sequence for human sFRP-1: alanine 174 instead of proline at this position (Melkonyan et al., Proc. Natl. Acad. Sci. USA 1997, 94:13636-13641). SEQ ID NO: 2 also differs by one amino acid from another reported sFRP-1 sequence (GenBank™ Accession No. NP_{—}003003; SEQ ID NO: 7) because it has 314 amino acids, while GenBank™ Accession No. NP_{—}003003 has 313 amino acids (this difference is due to an additional alanine residue at position 13 in SEQ ID NO: 2). The present invention encompasses compositions comprising and methods using all sFRP-1 proteins and polypeptides (and fragments thereof), and nucleotides encoding these polypeptides, including those shown in SEQ ID NO: 2, polypeptides having about at least 90% identity to SEQ ID NO: 2, GenBank™ Accession No. NP_{—003003}, and as reported in Melkonyan et al. (supra).

### Example 10

### Characterization of the Apoptotic Activity of hOB

Because the cloned full-length hOB sFRP gene was identical to human sFRP-1/FRP-1/SARP-2, the biological role of this gene in the hOB was investigated to determine whether the gene product regulated hOB cell viability and Wnt signaling.

hOB cells were seeded at 200,000 cells/well into 6-well dishes and incubated at 34° C. The next day, one set of plates were rinsed with PBS, trypsinized, and baseline cell number (and mean cell volume) was determined with a Coulter Multisizer as previously described in Bodine et al., J. Bone Miner. Res. 1996, 11:806-819, which is incorporated herein by reference. The other set of plates was placed at the non-permissive temperature of 39° C., and cell number was determined 6 days later (the medium was changed on day 3). The hOB-03-C5 cells, which are in the proliferative-stage of osteoblast differentiation, divided slowly at 39° C. and cell number increased by 60 to 80% after 6 days; this rate of cell division was similar to explant cultures of normal hOB cells (Bodine et al., Endocrinology 1996, 137:4592-4604). In contrast, the maturation-stage hOB-03-CE6 cells stopped dividing at the non-permissive temperature and cell number remained constant, while the pre-osteocytic hOB-01-C1 cells slowly died at 39° C. such that fewer than 40% of the cells remained alive after 6 days. As noted previously, overexpression of sFRP-1 in MCF-7 breast cancer cells accelerated the rate of cell death. Consistent with this observation, basal sFRP-1/FRP-1/SARP-2 mRNA expression dramatically increased in the pre-osteocyte hOB-01-C1 cells when compared to the proliferative hOB-03-C5 and mature hOB-03-CE6 cell lines. Thus, hOB-01-C1 accelerated cell death correlated with high basal expression of hOB sFRP mRNA in these cells.

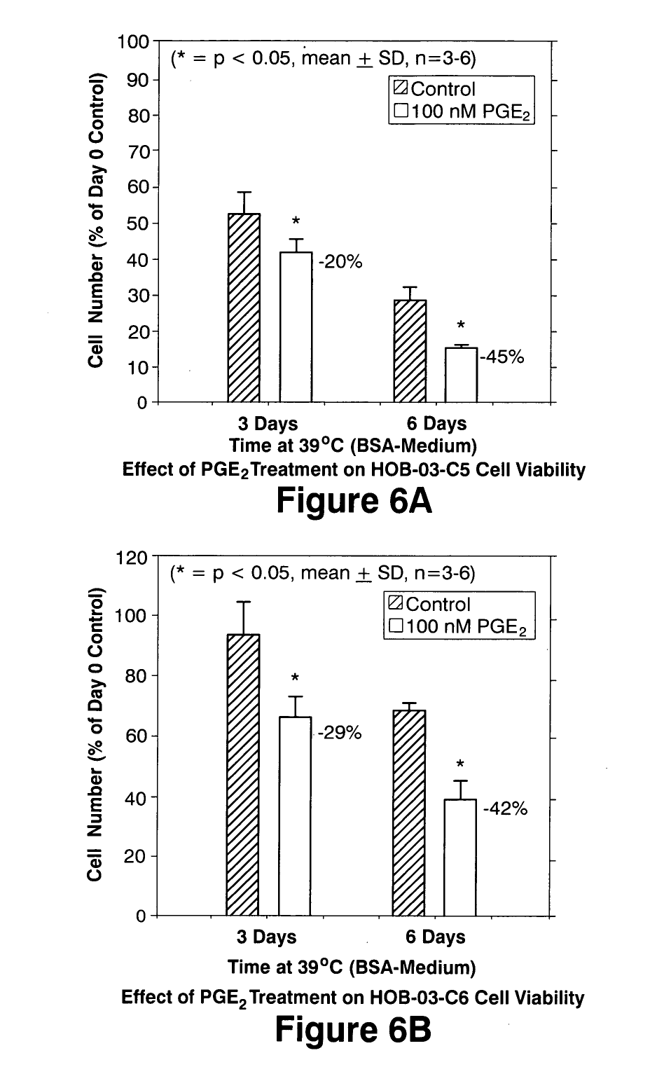

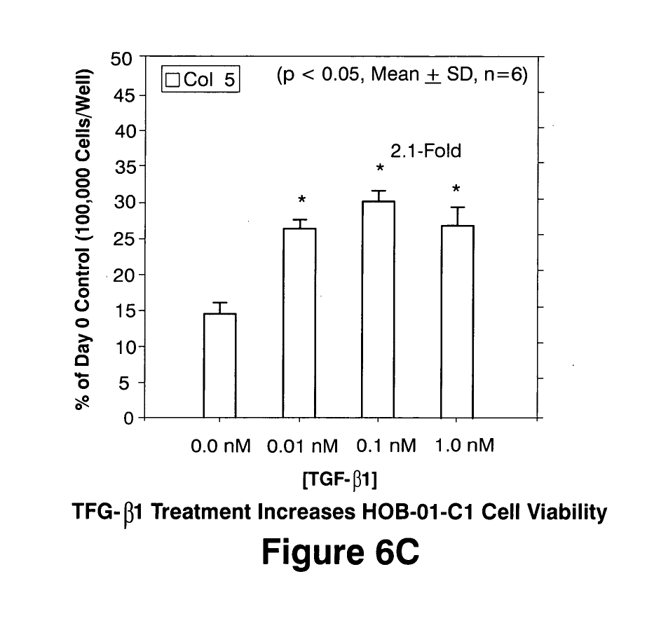

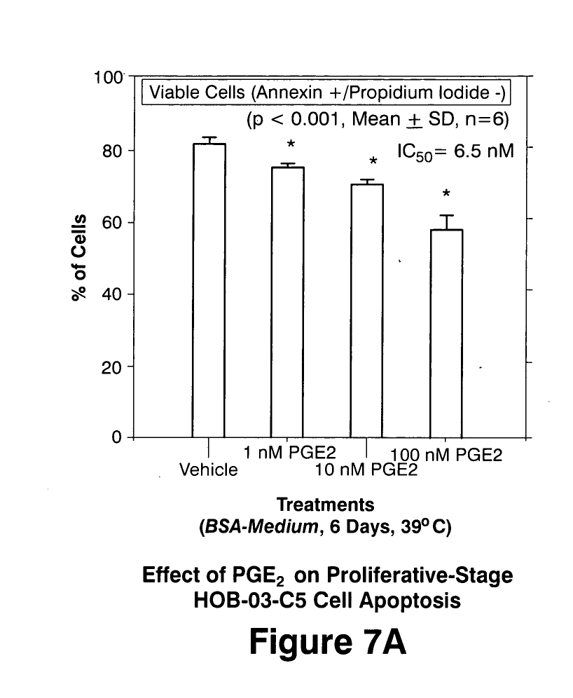

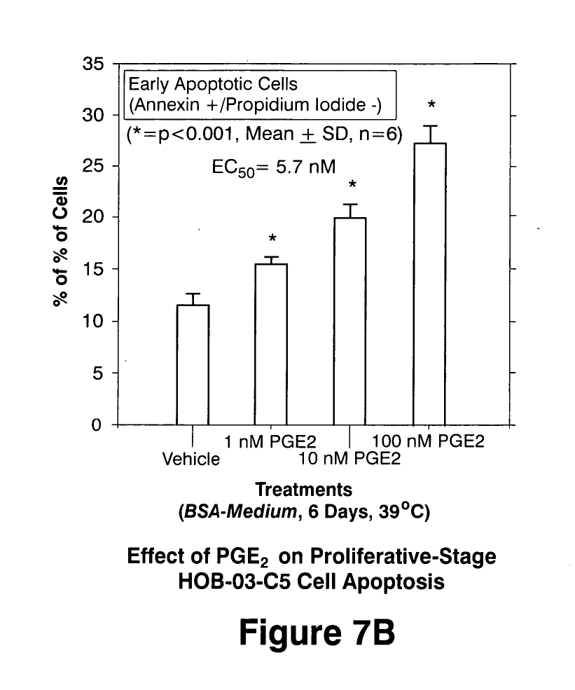

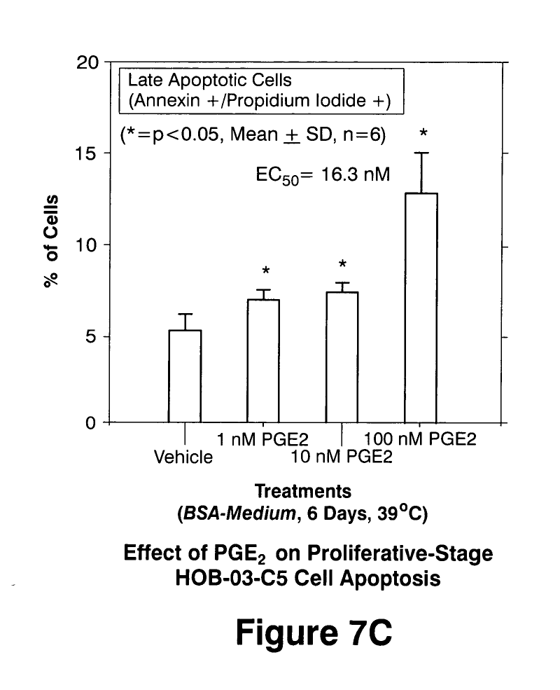

The hypothesis that up-regulation of sFRP-1/FRP-1/SARP-2 gene expression accelerates hOB cell death, while down-regulation of sFRP-1/FRP-1/SARP-2 gene expression suppresses cell death, was next examined. These results are depicted in FIGS. 6A-C. hOB-03-C5, hOB-03-CE6 or hOB-01-C1 cells were seeded with growth medium at about 200,000 cells per well into 6-well plates and incubated at 34° C. overnight. The next day, the 6-well plates were rinsed with PBS, placed in BSA-medium, and treated at 39° C. in the absence or presence of either 100 nM PGE2 (in order to up-regulate sFRP-1/FRP-1/SARP-2 steady-state mRNA levels; FIGS. 6A and B), or 0.01-1.0 nM TGF-β1 (in order to down-regulate sFRP-1/FRP-1/SARP-2 message levels; FIG. 6C). Incubating cells in serum-free medium is a common method to induce apoptosis (Melkonyan et al., Proc. Natl. Acad. Sci. USA 1997, 94:13636-13641), and the hOB-03-C5, hOB-03-CE6 and hOB-01-C1 cell lines all stopped dividing and gradually died under these conditions. However, the rate of cell death was significantly accelerated when the hOB-03-C5 and hOB-03-CE6 cells were treated with PGE2, such that over 40% fewer cells remained alive after 6-days of treatment. In contrast, treatment of the hOB-01-C1 cells with TGF-β1 increased cell viability about 2-fold in a dose-dependent manner. Treatment of the cells with PGE2 not only accelerated cell death, but also significantly reduced the mean cell volume by 10 to 20%. This observation was consistent with the induction of apoptosis, which is known to result in cytoplasmic blebbing, the loss of water and a decrease in cell volume (Mesner and Kaufmann, Advances in Pharmacology 1997, 41:57-88). Also consistent with the induction of apoptosis, PGE2-treatment of the hOB-03-C5 cells resulted in the generation of histone-associated DNA fragments. Finally, treatment of the hOB-03-C5 cells with PGE2 increased annexin V (a specific marker for apoptosis) binding to the cell as measured by flow cytometry (FIG. 7).

### Example 11

### Reversal of Cell Death Induction

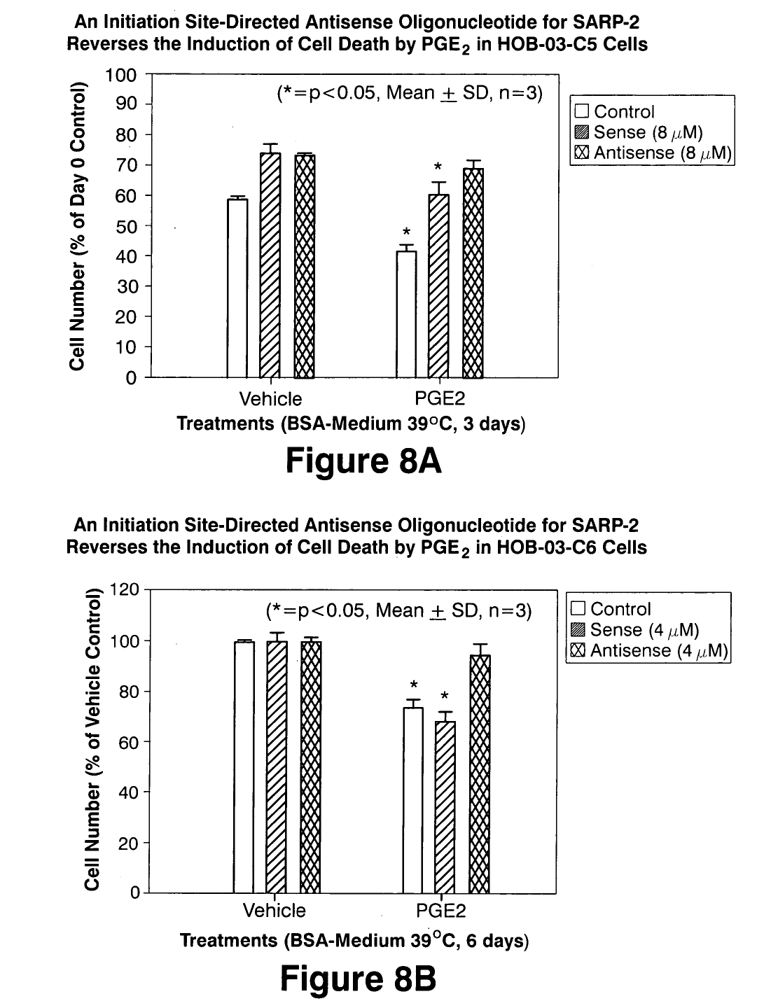

Reversal of cell death using an antisense oligonucleotide to sFRP-1/FRP-1/SARP-2 is demonstrated in FIG. 8 using hOB-03-C5 and hOB-03-CE6. These experiments were performed in a similar manner to the ones depicted in FIG. 6. However, for these experiments, the cells were co-treated with either vehicle control (i.e., 0.1% ethanol) or PGE₂ in the absence or presence of sense (control) or antisense initiation-site directed phosphorothioate oligonucleotides to human sFRP-1. The results are presented as either the % relative to the day 0 control (i.e., ˜200,000 cells per well of a 6 well plate) or as the % relative to the vehicle treated control. The results of this experiment indicate that co-treatment of the hOB cells with the antisense oligonucleotide to sFRP-1 reversed the ability of PGE₂ to accelerate the rate of cell death, while co-treatment with the sense (control) oligonucleotide has no effect on this process. In addition, co-treatment of the cells with PGE₂ and an anti-peptide antibody to sFRP-1 blocked the ability of PGE₂ to induce hOB cell death. The sequences for the sense and antisense oligonucleotides to human sFRP-1/SARP-2 are as follows:

| | | |
|---|---|---|
| Sense: | 5′-GGCATGGGCATCGGGCGC-3′ | (SEQ ID NO: 8) |
| Antisense: | 5′-GCGCCCGATGCCCATGCC-3′ | (SEQ ID NO: 9) |

### Example 12

### Overexpression of sFRP Accelerates hOB Cell Death

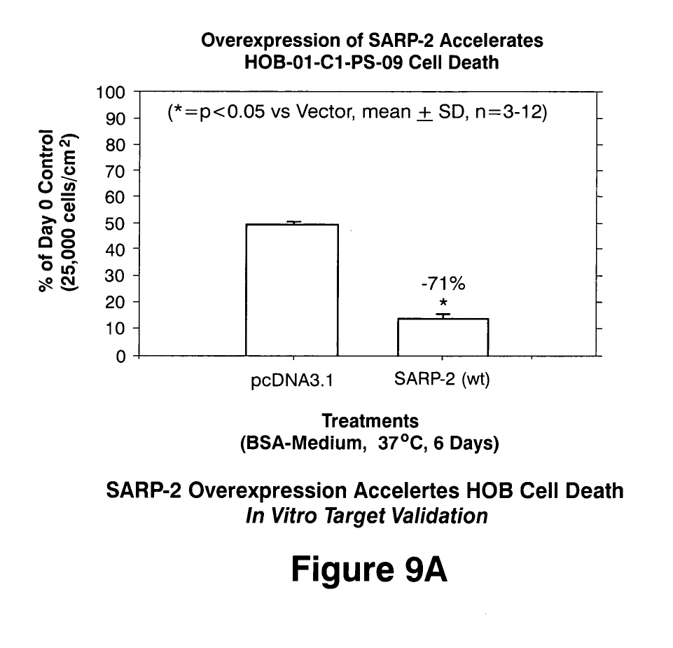

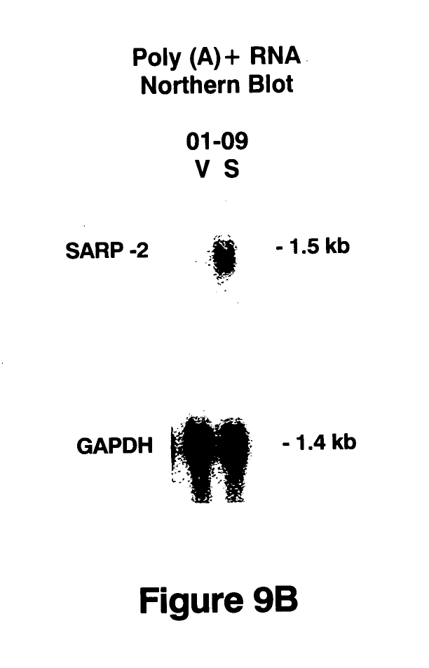

FIG. 9 shows the results of a cell viability experiment with the hOB-01-C1-PS-09 cells using the Coulter cell counter. For these experiments, the cells were stably transfected with either an hOB sFRP cDNA (i.e., sFRP-1/FRP-1/SARP-2) mammalian expression plasmid or the empty vector (i.e., pcDNA3.1, which is obtained from Invitrogen of Carlsbad, Calif.). Standard cloning techniques were used (See Ausubel et al., Short Protocols in Molecular Biology 1997, 3^{rd} edition, Wiley {New York}). The results are presented as the % cell viability of the day 0 control cells. The results of this experiment indicated that overexpression of sFRP-1/FRP-1/SARP-2 by the hOB cells accelerates the rate of cell death when compared to the empty vector which has no effect on this process. Autoradiograms of a Northern blot of total RNA isolated from the empty vector (v) or sFRP(S) expressing cells demonstrated that the hOB cells overexpressed the sFRP gene as expected. In addition, TaqMan quantitative RT-PCR analysis indicated that the sFRP overexpressing cells expressed 5-6 times more sFRP mRNA than the empty-vector expressing cell.

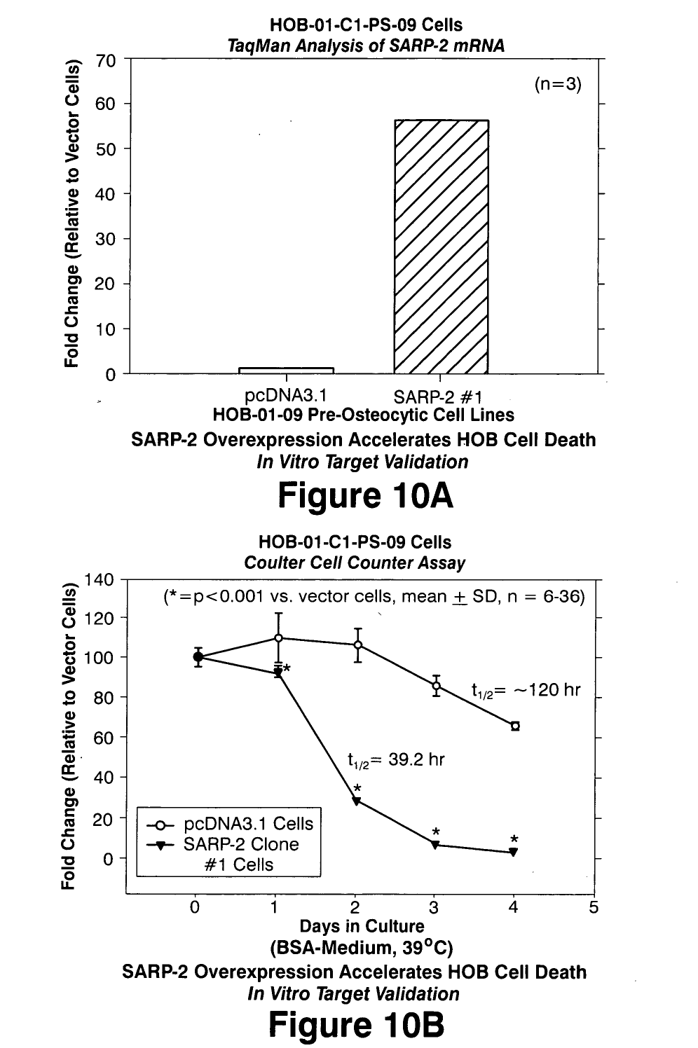

In order to improve the rate of cell death that was induced by sFRP-1/FRP-1/SARP-2, the overexpressing hOB-01-C1-PS-09 cells were subcloned and characterized (FIG. 10). TaqMan quantitative RT-PCR analysis indicated that one subclone (SARP-2 Clone #1) expressed 50-60-times more sFRP-1/FRP-1/SARP-2 mRNA than the pcDNA3.1 (empty vector) expressing cells. Likewise, the SARP-2 Clone #1 cells died at greatly accelerated rate (t_{1/2}=39.2 hr) in BSA-medium when compared to the empty vector expressing cells (t_{1/2}=˜120 hr).

### Example 13

### Effect of hOB sFRP on Wnt Activity

In order to determine if the up-regulation of sFRP-1/FRP-1/SARP-2 gene expression results in an antagonism of the Wnt signaling pathway, hOB-03-CE6 cells were treated with PGE₂ and the resulting cells probed with β-catenin monoclonal antibody. Overexpression of Wnt proteins in cells up-regulates a signaling protein known as β-catenin (reviewed in Moon et al., Cell 1997, 88:725-728; Barth et al., Curr. Opin. Cell Biol. 1997, 9:683-690; and Nusse, Cell 1997, 89:321-323). Moreover, overexpression of sFRP-1/FRP-1/SARP-2 in MCF-7 cells down-regulated β-catenin levels, which is consistent with an antagonism of Wnt activity (Melkonyan et al., Proc. Natl. Acad. Sci. USA 1997, 94:13636-13641). Therefore, hOB-03-CE6 cells were plated and treated with PGE₂ as described in Example 2, except that total cellular protein was extracted and a Western blot analysis for β-catenin was performed using a monoclonal antibody to the protein (Transduction Laboratories) as previously described (Bodine et al., Endocrinology 1996, 137:4592-4604; Melkonyan et al., Proc. Natl. Acad. Sci. USA 1997, 94:13636-13641). Consistent with up-regulation of sFRP-1/FRP-1/SARP-2 steady-state mRNA levels, treatment of hOB-03-CE6 cells with 100 nM PGE₂ for 24 hr down-regulated 13-catenin protein levels indicating an antagonism of Wnt activity. In addition, cotransfecting either human or rat sFRP-1/FRP-1/SARP-2 cDNA into either hOB-01-C1-PS-09 or hOB-02-C1-PS-02 cells down-regulated TCF-luciferase expression, which is an authentic measurement of Wnt signaling and β-catenin nuclear activity (e.g., Bafico et al., J. Biol. Chem. 1999, 274:16180-16187). Both human and rat sFRP-1/FRP-1/SARP-2, as well as human Frzb-1/FrzB/Fritz, suppressed TCF-luciferase activity in the hOB cells.

All together, these observations suggest that a Wnt protein(s) prolongs the life of human osteoblasts in vitro and that antagonism of Wnt signaling by sFRP-1/FRP-1/SARP-2 promotes osteoblast cell death. Thus, an inhibitor of sFRP-1/FRP-1/SARP-2 function may increase osteoblast/pre-osteocyte survival and therefore enhance bone formation in vivo.

Using several methods to characterize Wnt expression in the hOB cells (e.g., RT-PCR, GeneChip analysis and cDNA cloning), we have evidence that these cell lines express, to varying degrees, Wnt-2, 2B/13, -3, -4, -5A, and -11. Anyone or all of these Wnts could be involved in prolonging hOB cell life. Wnt-2B/13 is also known as Wnt-x.

### Example 14

### Use of hOB sFRP in a Screening Method for Anabolic Agents

A new screening paradigm for an anabolic bone agent using sFRP has been designed. As outlined in FIG. 11, this screening paradigm uses the hOB cells and sFRP-1/FRP-1/SARP-2 to identify compounds that are capable of preventing or slowing osteoblast cell death. Such compounds act by blocking the ability of sFRP-1/FRP-1/SARP-2 to accelerate hOB cell death. These compounds bind to sFRP-1/FRP-1/SARP-2 and prevent it from binding a Wnt protein, or they may bind to a Wnt and prevent it from binding to sFRP-1/FRP-1/SARP-2. If sFRP-1/FRP-1/SARP-2 has activities that are independent of Wnt-binding (e.g., binding to a cell surface receptor), then these compounds could also act by preventing this Wnt-independent function as well.

For the initial assay of the screening paradigm outlined in FIG. 11, compounds are incubated with an hOB cell line and sFRP-1/FRP-1/SARP-2. This assay could use purified or partially purified sFRP-1/FRP-1/SARP-2 protein, or conditioned-media or cell extracts that contained sFRP-1/FRP-1/SARP-2. The hOB cell line could be one that naturally expressed high basal levels of sFRP-1/FRP-1/SARP-2 (e.g., hOB-01-C1 cells), that transiently or stably overexpressed sFRP-1/FRP-1/SARP-2 (e.g., hOB-01-C1-PS-09 cells), or that stably or naturally expressed sFRP-1/FRP-1/SARP-2 in a conditional manner (e.g., hOB-03-C5 cells treated with PGE₂). As a measurement of hOB cell death, assays quantify cell number (e.g., MTT or MTS dye-conversion or CyQuant DNA fluorescence) or apoptosis (e.g., DNA fragmentation or annexin V binding) could be used. CyQuant kits were purchased from Molecular Probes (Eugene, Oreg.).

An example of a high-throughput screening assay (HTS) for sFRP-1/FRP-1/SARP-2 inhibitors is depicted in FIG. 12. For this assay, either empty vector (pcDNA3.1) or sFRP-1/FRP-1/SARP-2 (SARP-2 #1) stable overexpressing hOB-01-C1-PS-09 cells were seeded at 5000 cells per well into 96-well plates using growth medium. After a 6 hr incubation at 34° C., the wells were rinsed with PBS and incubated in BSA-medium at 39° C. for 3 days. At the end of the incubation, the wells were rinsed again with PBS and then assayed for DNA content using the CyQuant DNA fluorescence assay (Molecular Probes). When compared to the empty vector cells, the sFRP-1 overexpressing cells died faster at 39° C. such that after 3 days, only 20-30% of the cells were still alive. In contrast, 50-60% of the empty vector cells survived the incubation. When the sFRP-1 overexpressing cells were treated with an antipeptide antisera (AS) generated to amino acids 217-231 of sFRP-1/FRP-1/SARP-2, 50-60% of the cells were alive after 3 days. This indicates that inhibition of sFRP-1/FRP-1/SARP-2 protein function prevents it from accelerating hOB cell death. As controls, the pre-immune serum had no effect on the sFRP-1 overexpressing cells, and neither the pre-immune nor the immune sera affected the empty vector expressing cells.

Compounds that blocked hOB cell death induced by sFRP-1/FRP-1/SARP-2 would then move on to additional in vitro assays. These assays measure the ability of these compounds to block hOB cell death in an sFRP-1/FRP-1/SARP-2-dependent or independent manner, and they would also determine the potency and efficacy of these compounds for these effects. Additional assays are designed to determine the cell selectivity of these compounds for these effects (e.g., by using MCF-7 or other cells), as well as the specificity of these compounds for sFRP-1/FRP-1/SARP-2 versus another member of the sFRP/SARP family (e.g., FrzB/Fritz, SARP-1, or SARP-3). Additional assays could also be used to determine if these compounds regulate down-stream signaling events involved in apoptosis (e.g., caspase activity) or Wnt activity (e.g., β-catenin levels and function via the TCF-luciferase assay). Finally, compounds that exhibited appropriate activities in these in vitro assays would then be used in a variety of animal models for bone formation, osteopenia, or osteoporosis (e.g., ovariectomized rats or mice). A compound that inhibited osteoblast/osteocyte apoptosis would conceivably be an anabolic bone agent by prolonging the lives of these cells and thereby either increasing the amount of bone matrix that is synthesized and mineralized and/or maintaining the integrity of the bone.

It is clear that the invention may be practiced otherwise than as particularly described in the foregoing description and examples. Numerous modifications and variations of the present invention are possible in light of the above teachings and therefore are within the scope of the appended claims.

### Example 15

### Development of sFRP-1 Knock-Out Mice

As stated in Example 14, a compound that inhibited osteoblast/osteocyte apoptosis would conceivably be an anabolic bone agent by prolonging the lives of these cells and thereby either increasing the amount of bone matrix that is synthesized and mineralized and/or maintaining the integrity of the bone. In order to test this hypothesis and determine if sFRP-1/FRP-1/SARP-2 affects the skeleton, sFRP-1 −/− mice were prepared (See Wattler et al., BioTechniques 1999, 26:1150-1160). Deleting the sFRP-1/FRP-1/SARP-2 gene from mice would be akin to inhibiting its function with a drug, and this process allows us to validate this gene/protein as a potential drug target for osteoporosis.

The sFRP-1 knock-out mice were generated by substituting exon 1 (which encodes amino acids 1-181 of the sFRP-1 polypeptide) of the mouse sFRP-1 gene with a β-galactosidase reporter gene/neomycin drug resistance gene expression cassette (a 5.2 Kb cassette). The sFRP-1 mice were produced by Lexicon Genetics, Inc. (The Woodlands, Tex.). Exon 1 was chosen because it encodes most of the protein, including the entire CRD. This exon was replaced with a LacZ/MC1-Neo selection cassette so that sFRP-1 promoter activity could be followed with f-galactosidase expression in −/− mice.

The targeting vector was derived from the Lambda KOS system (Wattler et al., Biotechniques 1999, 26:1150-1160). Genomic clones were isolated from a Lambda KOS library by PCR using the exon 1-specific forward primer (5′-CTGAGGCTGTGCCACAACG-3′; SEQ ID NO: 10) and the reverse primer (5′-CATGACCGGCTCGCACGAG-3′; SEQ ID NO: 11). A yeast selection cassette containing the URA3 marker was generated by PCR using a sense primer containing the gene-specific sequence (5′-GCAGCGGGACGCGCGCGTGAAGGCAGCGTG-3′; SEQ ID NO: 12) and an antisense primer containing the gene-specific sequence (5′-GGGGTTCGCGGGCGTGGGAAGGCATACCCT-3′; SEQ ID NO: 13). This marker was introduced into the genomic clone by yeast recombination and resulted in the deletion of 1176 bp of exon 1 that was subsequently replaced with a LacZ/MC1-Neo selection cassette. The Not I linearized vector was electroporated into 129 Sv/Evbrd (LEX1) embryonic stem (ES) cells. G418/FIAU resistant ES-cell clones were isolated and analyzed for homologous recombination using Southern analysis. The 5′ probe was a 510 bp PCR fragment derived from the sense primer (5′-ATGTGTATCTTGAGTTGGTATC-3′; SEQ ID NO: 14) and the antisense primer (5′-CATAATACTTGCAAATTGATGC-3′; SEQ ID NO: 15). Use of this probe on Eco RI digested genomic DNA produced a 12 Kb wild type and 10 Kb mutant band. The 3′ probe was a 542 bp PCR fragment derived from the sense primer (5′-CAACATAGCACTACATCTTCG-3′; SEQ ID NO: 16) and the antisense primer (5′-GGCCAACGCTGAAGCCAG-3′; SEQ ID NO: 17). Use of this probe on Bam HI-digested genomic DNA produced a 13 Kb wild type and 9.5 Kb mutant band. Four targeted ES-cell clones were identified and injected into C57BL/6 (albino) blastocysts. The resulting chimeras were mated to C57BL/6 (albino) females to generate sFRP-1+/− animals. Subsequent intercrosses of sFRP-1+/− mice were performed to produce +/+, +/− and −/− animals. Additional matings of these mice were equally successful and produced litters of similar size.

Animals were grouped by sex, age and genotype with ad libitum access to filtered chlorinated water from the municipal water supply and a standard rodent diet containing 0.9% (w/v) calcium and 0.7% (w/v) phosphorous (Purina No. 5001 Rodent Diet). Environmental conditions were set to maintain 22+/−3° C., 40+/−15% humidity, and 12-hour alternating light/dark cycle. Animal health observations and environmental parameters were monitored daily. As expected, sFRP-1+/− mice carried both alleles. Weanlings were ear- or tail-biopsied at 3 weeks of age. For this analysis, DNA was extracted for 90 min at 56° C. in a PE 9600 thermocycler (PE Applied Biosystems, Foster City, Calif.) with lysis buffer that was composed of GeneAmp PCR buffer (PE Applied Biosystems), 2.5 mM MgCl₂, 0.5% (v/v) Tween-20 and 100 μg/ml of proteinase K (GIBCO Invitrogen, Gaithersburg, Md.). The lysis reaction was terminated by incubation of the samples for 10 min at 99° C., followed by cooling to 4° C., vortexing and centrifugation.

Mice were initially genotyped using conventional PCR with the following primer sets: sFRP-1 (forward 5′-GGCAGCCCCGACGTCGCCGAGCAAC-3′ (SEQ ID NO: 18), reverse 5′-CCTTGGGGTTAGAGGCTTCCGTGG-3′ (SEQ ID NO: 19); a 379 bp fragment); β-galactosidase (forward 5′-ACGGCATGGTGCCAATGAATCGTCTG-3′ (SEQ ID NO: 20), reverse 5′-CAAATAATATCGGTGGCCGTGGTGTC-3′ (SEQ ID NO: 21); a 212 bp fragment). The 50 μL PCR reaction contained PCR buffer, GC Melt and Advantage-GC cDNA Polymerase Mix (Clontech, Palo Alto, Calif.), 1.0 μM of each primer, 0.2 mM dNTPs and 2.0 μL of lysate. After 30-35 cycles at an annealing temperature of 68° C., the PCR products were resolved by electrophoresis on 1.5% agarose gels and visualized with ethidium bromide.

Genotyping was later performed using real-time PCR with an ABT PRISM 7700-sequence detection system (PE Applied Biosystems) as described by the manufacturer. The following Taqman primers and probe sets were used: sFRP-1 (forward 5′-GCCACAACG TGGGCTACAA-3′ (SEQ ID NO: 22), reverse 5′-ACCTCTGCCATGGTCTCGTG-3′ (SEQ ID NO: 23), probe 6-FAM-5′-AGATGGTGCTGCCCAACCTGCTG-TAMRA-3′ (SEQ ID NO: 24)); β-galactosidase (forward 5′-CTGCTGATGAAGCAGAACAACTTT-3′ (SEQ ID NO: 25), reverse 5′-GCGTGTACCACAGCGGATG-3′ (SEQ ID NO: 26), probe VIC-5′-CGCCGTGCGCTGTTCGCATTA-TAMRA-3′ (SEQ ID NO: 27)).

To confirm that −/− mice no longer expressed sFRP-1, Northern blot analysis was performed with poly A(+) RNA isolated from kidneys of +/+ and −/− animals at 16-18 weeks of age. As expected, this tissue showed high levels of sFRP-1 mRNA expression in +/+ females and males, whereas −/− mice did not express the message.

Because the Northern blot analysis described above was performed with a probe that hybridized to the CRD, we also isolated total RNA from calvarial bones of 32 week old mice and measured sFRP-1 message levels by real-time RT-PCR analysis using primer and probe sets specific for either the 5′-region (CRD) or the 3′-region (netrin domain). This analysis confirmed that sFRP-1−/− mice did not express either portion of the gene, since +/+ calvaria expressed mRNA as measured by both primer/probe sets, while −/− calvaria did not express message by these measurements.

This quantification of sFRP-1 mRNA was performed with total RNA using real-time RT-PCR on an ABT PRISM 7700-sequence detection system as described by the manufacturer. The following Taqman primers and probe sets were used: sFRP-1 CRD (forward 5′-GCCACAACG TGGGCTACAA-3′ (SEQ ID NO: 22), reverse 5′-ACCTCTGCCATGGTCTCGTG-3′ (SEQ ID NO: 23), probe 6-FAM-5′-AGATGGTGCTGCCCAACCTGCTG-TAMRA-3′ (SEQ ID NO: 24)); sFRP-1 netrin domain (forward 5′-CGCTTGTGCTGTTCCTGAAG-3′ (SEQ ID NO: 28), reverse 5′-CGGCCCATGATGAGAAAGTT-3′ (SEQ ID NO: 29), probe 6-FAM-5′-TGCCGACTGTCCCTGCCACCA-TAMRA-3′ (SEQ ID NO: 30)). The sFRP-1 results were normalized to 18S ribosomal RNA levels using VIC-probe reagents from PE Applied Biosystems (part # 4308329), and mRNA levels were calculated using the Standard Curve Method as described by Applied Biosystems in User Bulletin #2.

### Example 16

### Deletion of sFRP-1 Does Not Affect Nonskeletal Tissues, Skeletal Morphology or Cortical Bone

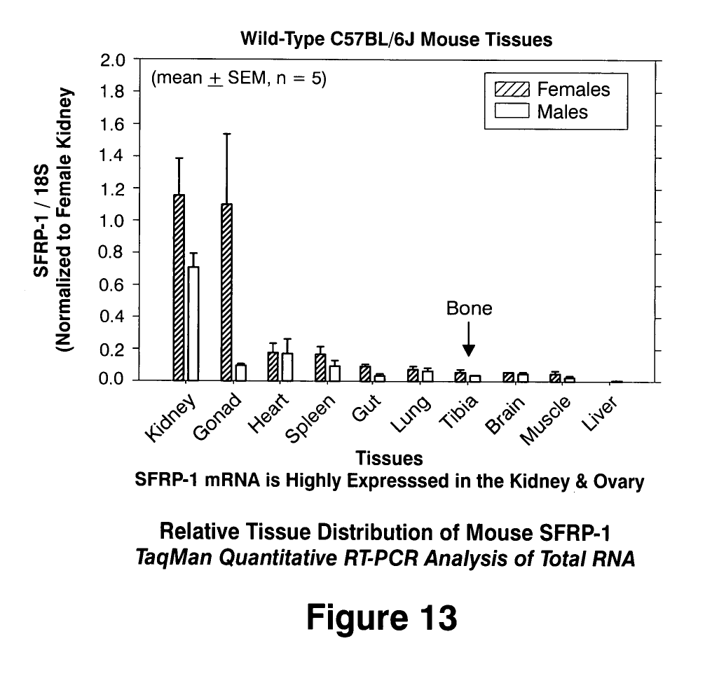

In order to determine the relative tissue distribution of sFRP-1 mRNA in wild-type mice, we isolated total RNA from a variety of tissues and then quantified the message using TaqMan real-time RT-PCR analysis. As shown in FIG. 13, although sFRP-1 mRNA is expressed in bone (tibia), it is expressed at much higher levels in kidneys and ovaries from the mice. Despite this tissue distribution, the −/− mice are fertile and viable and have been kept alive for over 18 months with no overt phenotype. No clinically relevant changes have been observed in many non-skeletal parameters including the following: body and organ weights: serum calcium, phosphorus, bone-alkaline phosphatase, osteocalcin levels and type I collagen C-terminal telopeptides (these two were determined by ELISA using Rat-Mid Osteocalcin and RatLaps kits manufactured by Osteometer BioTech A/S (Herlev, Denmark)); urinary deoxy-pyridinoline cross-link levels; total body BMD, bone mineral content (BMC) and % body fat (except for 40-week-old male −/− mice that have 22% less body fat than +/+ controls); and cortical BMD. The overall skeletal morphology of sFRP-1−/− mice as determined by radiographic analysis was normal. Using an XCT Research peripheral Quantitative Computed Tomography densitometer (pQCT; Stratec Medizintechnik, Pforzheim, Germany) to measure volumetric bone mineral density (vBMD; mg/cm³), we did not observe changes up to 41 weeks of age in cortical bone parameters such as femoral diaphyseal BMD, thickness, or periosteal and endosteal circumference, and femur length was not affected. Volumetric BMD and cortical bone measurements were evaluated as described (Turgeman et al., J. Cell. Biochem. 2002, 86:461-474; Babij et al., Gene J. Bone Miner. Res. 2003, 18:960-974).

These observations suggest that sFRP-1 is redundant in most non-skeletal tissues and is not required for most developmental and physiological processes in mice. Furthermore, these results indicate that deletion of sFRP-1 does not affect skeletal development, cortical bone formation, or bone turnover and metabolism, although it may suppress adipogenesis in aged male mice. The observation that sFRP-1−/− males have diminished percent body fat supports a previous study showing that activation of the canonical Wnt pathway in 3T3-L1 preadipocytes in vitro inhibits adipogenesis (Ross et al., Science 2000, 289:950-953).

### Example 17

### Deletion of sFRP-1 Results in Increased Trabecular Bone Formation

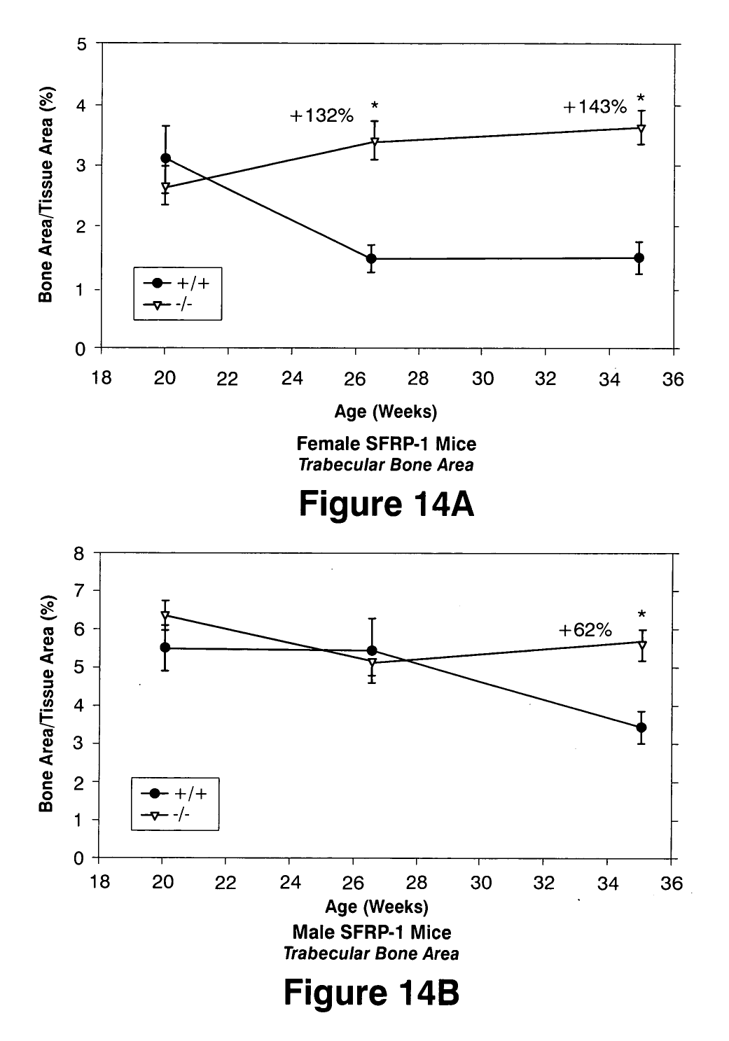

Histology of proximal femurs from 35 week old mice indicated that deletion of sFRP-1 increased femoral trabecular bone area by 143% in −/− females (FIG. 14A) and by 62% in −/− males (FIG. 14B) when compared to +/+ controls. At this age, femurs of sFRP-1+/+ mice contained relatively little trabecular bone due to age-dependent loss of the tissue. Histomorphometry of proximal femurs for a review of this technique, see P. J. Meunier 1995 Bone Histomorphometry, in Osteoporosis: Etiology, Diagnosis, and Management, 2nd edition, B. L. Riggs & L. J. Melton III, editors, Lippincott-Raven, Philadelphia, pages 299-318)) from females at age 20-35 weeks indicated that trabecular bone area was similar between sFRP-1+/+ and sFRP-1−/− mice at 20 weeks of age (FIGS. 14A and B). However, between 20-35 weeks in females and 27-35 weeks in males, +/+ mice lost bone area, while −/− animals retained trabecular bone area (FIGS. 14A and B). Similarly, between 20-35 weeks in females and 27-35 weeks in males, +/+ mice lost trabecular bone mineralized area, while −/− animals trabecular bone mineralized area was also observed to be 62% higher in males and 143% higher in the females. These results indicate that deletion of sFRP-1 delays the onset of age-dependent femoral trabecular bone loss.

In order to determine if ablation of sFRP-1 affects trabecular bone formation, we measured the mineral apposition rate (MAR) using in vivo calcein double labeling in 35 week old mice. Static and dynamic histomorphometry measurements of the femur were made using the R&M Biometrics Inc. Bioquant Image Analysis System (Bioquant Image Analysis Corp., Nashville, Tenn.) as previously described (Babij et al., Gene J. Bone Miner. Res. 2003, 18:960-974). For a review of this technique, see Meunier Bone Histomorphometry, in Osteoporosis: Etiology, Diagnosis, and Management 1995, 2nd edition, B. L. Riggs & L. J. Melton III, eds., Lippincott-Raven, Philadelphia:299-318.

Deletion of sFRP-1 increased trabecular MAR of the distal femur by 32% in females, although it was not altered in males. Because MAR is a measurement of the amount of bone matrix produced by individual osteoblast teams (Aaron et al., Journal of Histochemistry & Cytochemistry 1984, 32:1251-1261), these findings indicate that ablation of sFRP-1 enhances osteoblast activity in at least females. The reasons that MAR did not significantly increase in male sFRP-1−/− mice is not clear, but may be related to the smaller gains in trabecular BMD and bone volume seen in males at this skeletal site.

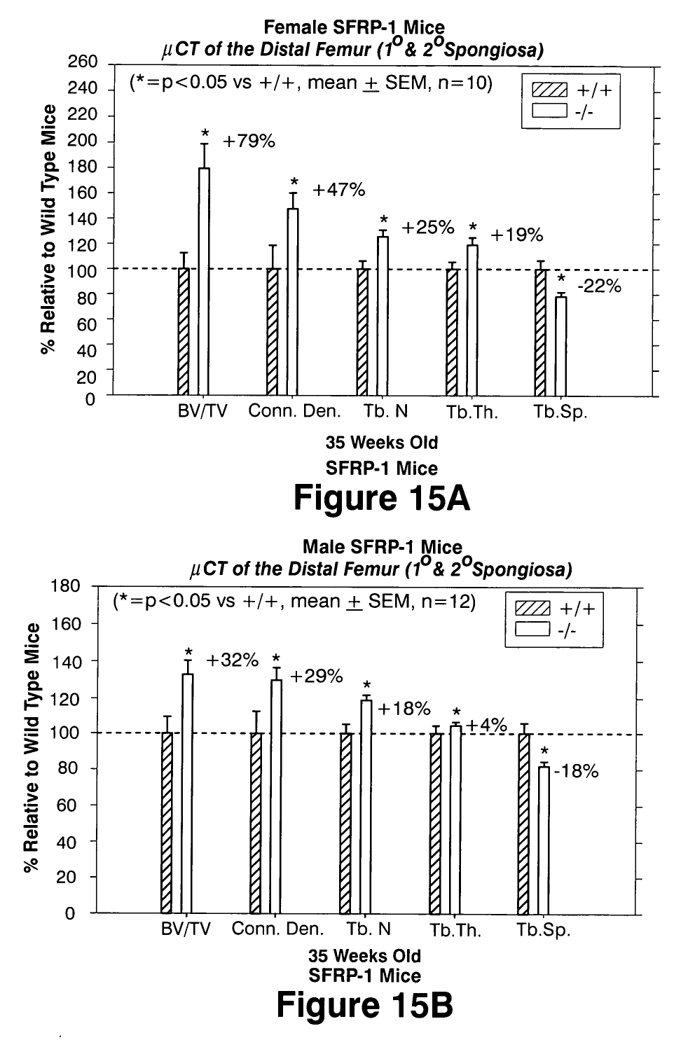

In order to confirm the histology findings, high-resolution micro-computed tomography (μCT) was performed on the distal portions of femurs from the mice (for a review of this technique, see Genant et al., Bone 1999, 25:149-152; Genant et al., Hormone Research (supplemental 1) 2000, 54:24-30; Jiang et al., J. Musculoskel. Neuron. Interact. 2000, 1:45-51; and Odgaard, Bone 1997, 20:315-328). Deletion of sFRP-1 increased trabecular bone volume by 79% in −/− females and by 32% in −/− males when compared to +/+ animals measured at 35 weeks of age (FIGS. 15A and B). Detailed analysis of these data demonstrated that removal of sFRP-1 not only led to an increase in trabecular bone volume (BV/TV), but also resulted in increased connectivity density (29-47%), trabecular number (18-25%), and trabecular thickness (4-19%) when compared to sFRP-1+/+ controls (FIGS. 15A and B). Moreover, as expected from these results, trabecular separation was decreased by 18-22% in sFRP-1−/− animals. These data demonstrate that loss of sFRP-1 leads to an enhancement of trabecular bone quality.

The μCT analysis also confirmed the temporal nature of the bone phenotype in sFRP-1−/− mice. For example, in −/− females, differences in femoral trabecular bone parameters like BV/TV began to occur at the 26-27 week time point and became optimal by 35 weeks of age. However, by 46-47 weeks of age, the differences between +/+ and −/− animals were no longer apparent. Likewise, when analyzed prior to 20 weeks of age, the femoral trabecular bone parameters of +/+ and −/− mice were also similar. Thus, deletion of sFRP-1 did not affect peak trabecular bone mass that occurs in mice at ˜2 months of age, but instead delayed by ˜20 weeks the age at which senile bone loss occurs.

μCT20 or 40 (Scanco Medical AG, Basserdorf, Switzerland) was used to evaluate trabecular bone volume fraction and microarchitecture of the distal femur as previously described (Turgeman et al., J. Cell. Biochem. 2002, 86:461-474; Babij et al., Gene J. Bone Miner. Res. 2003, 18:960-974).

We also examined the vertebrae for changes in trabecular bone parameters. Histomorphometry of vertebrae from 39-40 week old females indicated that deletion of sFRP-1 decreased trabecular spacing by 43%. Trabecular bone volume and trabecular number were also increased by ˜60% in the female sFRP-1−/− mice, but the differences between +/+ and −/− did not achieve significance, perhaps due to the small sample size (n=6/group). A similar pattern was also seen in males, although the differences between sFRP-1−/− mice and sFRP-1+/+ mice were smaller. These results indicate that deletion of sFRP-1 in mice leads to increases in trabecular bone parameters at multiple skeletal sites between the ages of 20-40 weeks.

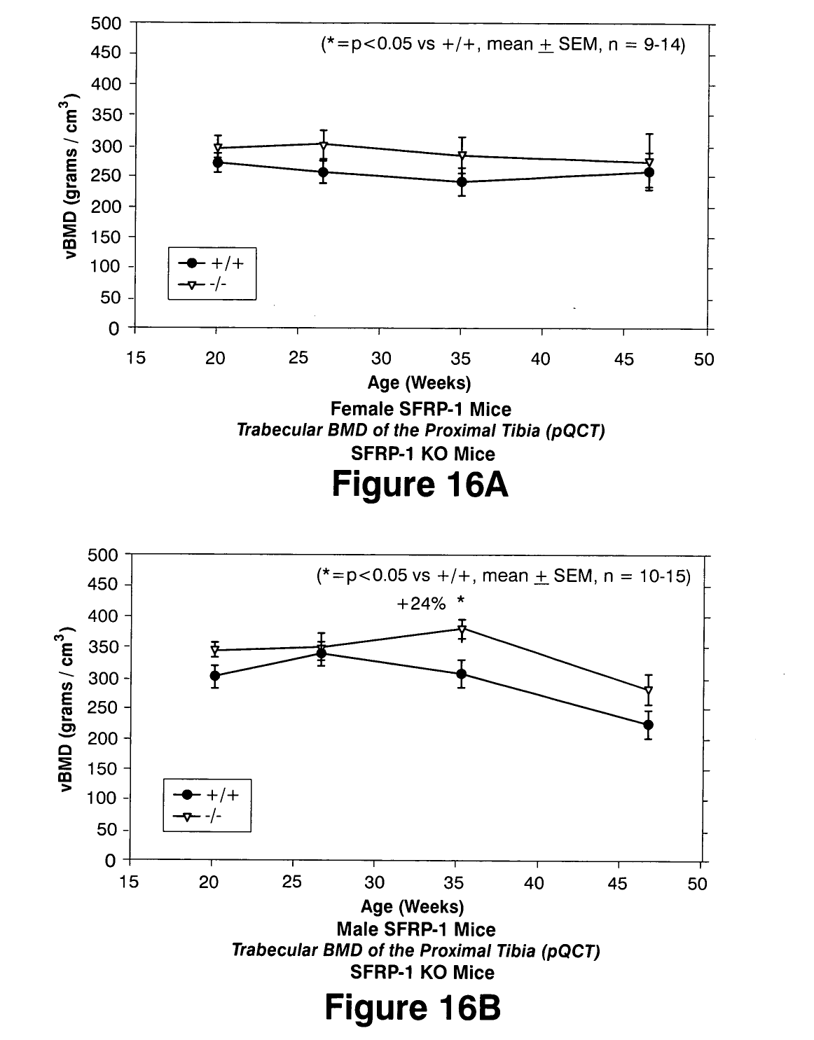

Volumetric trabecular bone mineral density (vBMD) of the tibia was also higher in the −/− mice as measured by pQCT (for a review of this technique, see Genant et al., Hormone Research (supplement 1) 2000, 54:24-30). As shown in FIGS. 16A and B, trabecular vBMD of the proximal region of the tibia increased by as much as 24% in the −/− mice when compared to the +/+ controls. Again, the largest difference between the −/− and +/+ animals was observed at 35 weeks of age.

Although the −/− mice exhibit large increases in trabecular bone parameters, the −/+ mice do not. Micro-CT analysis of femurs from 32-33 week old female mice indicate that the −/− animals have a 54-69% increase in distal femur BV/TV and Conn. Den. when compared to the +/+ controls, while the −/+ animals showed no change in these parameters. These results indicate that complete ablation of the sFRP-1 gene is required to generate increases in trabecular bone formation, and that one copy of the gene is sufficient to prevent these gains.

### Example 18

### Deletion of sFRP-1 Decreases Osteoblast and Osteocyte Apoptosis but Enhances Osteoprogenitor Differentiation

Our in vitro studies indicated that overexpression of sFRP-1 in human osteoblasts and osteocytes accelerated programmed cell death or apoptosis. In order to determine if osteoblast and osteocyte apoptosis was altered in sFRP-1−/− mice, we performed TUNEL staining of calvaria from 32-33 week old females. Deletion of sFRP-1 led to a 48% and 56% decrease in the number of apoptotic osteoblasts and osteocytes, respectively. This decrease in apoptosis resulted in an 18% increase in calvarial thickness and a 5% increase in osteocyte number. When the % apoptotic osteocytes per mm² was calculated, sFRP-1−/− females exhibited a 65% decrease in this parameter when compared to sFRP-1+/+ controls. Thus, while sFRP-1 is certainly not the only regulator of apoptosis in murine osteoblasts and osteocytes, it seems to contribute to about half of this process.

The TUNEL (Roche Diagnostics, Indianapolis, Ind.) method was use to detect cell apoptosis in paraffin-embedded mouse parietal bone (5 μm thick) as previously described (Zhao et al., J. Clin. Inv. 2000, 106:941-949; Babij et al., Gene J. Bone Miner. Res. 2003, 18:960-974). Cells undergoing apoptosis (TUNEL-positive) were identified using fluorescence microscopy.

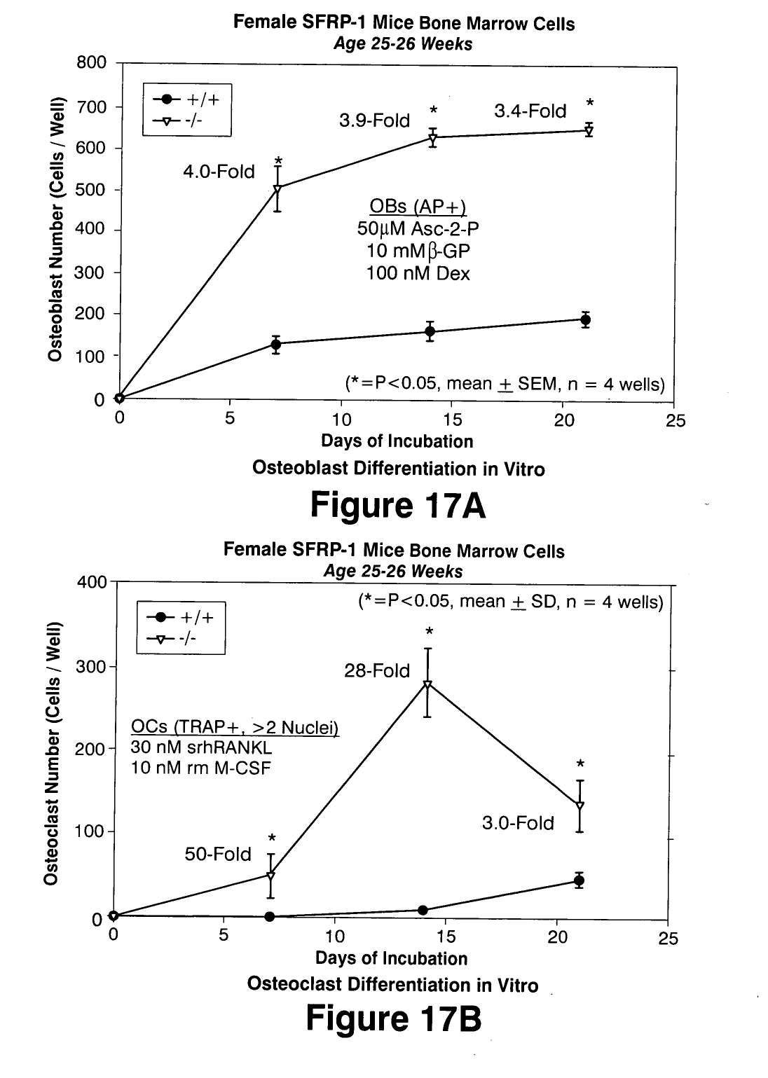

In order to determine if osteogenesis was altered by deletion of sFRP-1, we isolated bone marrow from +/+ and −/− mice and performed an in vitro differentiation assay. Incubation of bone marrow from 25-26 week old sFRP-1+/+ females for 21 days with ascorbic acid, β-glycerol phosphate and dexamethasone led to an increase in the number of ALP+ cells (FIGS. 17A and B). However, this effect was enhanced 3-4 fold with bone marrow from sFRP-1−/− mice. In addition, β-galactosidase staining of −/− cultures after 12 and 16 days of incubation indicated that sFRP-1 promoter activity also increased with advancing osteoblast differentiation, which confirms the in vitro observations for the elevated expression of sFRP-1 mRNA in hOB cells as a function of their differentiation. Furthermore, this increase correlated with an up-regulation of ALP staining in sFRP-1−/− cultures, as well as a marked enhancement of von Kassa staining when compared to sFRP-1+/+ cultures. This increase in ALP staining and mineralization in the sFRP-1−/− cultures confirms the observations for enhanced osteoblast function that were demonstrated by the elevation in trabecular MAR in the −/− mice.

These results demonstrate that sFRP-1 expression increases with advancing osteoblast maturation, and that deletion of this gene in vivo enhances osteoprogenitor differentiation and mineralization in vivo.

Hausler et al. have presented preliminary results showing that sFRP-1 binds to and blocks the effects of receptor activator of nuclear factor-κB ligand (RANKL) and tumor necrosis factor (TNF)-α on in vitro osteoclastogenesis (Hausler et al., J. Bone Miner. Res. 2001, 16:S153; Hausler et al., J. Bone Miner. Res. 2002, 17:S349). RANKL, along with monocyte-colony stimulating factor (M-CSF), is required for osteoclast differentiation (Teitelbaum, Science 2000, 289:1504-1508). In order to determine if osteoclastogenesis was also altered by deletion of sFRP-1, we performed an in vitro osteoclast differentiation assay with bone marrow from the mice. Incubation of bone marrow from 25-26 week old sFRP-1+/+ females for 21 days with soluble human RANKL and murine M-CSF led to an increase in the number of tartrate-resistant acid phosphatase (TRAP) positive, multinuclear cells (FIG. 17B). However, this effect was enhanced 3-50 fold with bone marrow from sFRP-1−/− mice. Although deletion of sFRP-1 resulted in a stimulation of osteoclastogenesis in vitro, as noted above, we did not observe any changes in bone resorption markers in sFRP-1−/− mice. Moreover, the phenotype of these animals is most consistent with an increase in osteoblastic function and not an increase in osteoclastic activity that typically results in osteoporosis (Peacock et al., Endocr. Rev. 2002, 23:303-326). Consequently, it appears that other regulators of osteoclast differentiation (e.g., osteoprotegerin) are able to compensate for loss of sFRP-1 in vivo (Teitelbaum, Science 2000, 289:1504-1508; Goltzman, Nat. Rev. Drug Disc. 2002, 1:784-796).

Isolation and culture of bone marrow derived mesenchymal stem cells (MSCs) and hematopoietic stem cells were performed as previously described (Gazit et al., J. Cell. Biochem. 1998, 70:478-488; Gazit et al., J. Cell. Biochem. 1999, 73:379-389). Cells were cultured in DMEM (high glucose) containing 1.0 mM GlutaMAX-1, 1.0% penicillin-streptomycin (all from GIBCO Invitrogen) and 15.0% heat-inactivated charcoal-stripped fetal bovine serum (HyClone Laboratories, Inc., Logan, Utah) at 37° C. in a 5.0% CO₂/95.0% humidified air incubator (Form a Scientific, Marietta, Ohio). Supplements for osteoblast differentiation included 10.0 mM β-glycerol phosphate (β-GP) (Sigma-Aldrich, St. Louis, Mo.), 10-7 M dexamethasone (Dex), 50.0 μM ascorbate-2-phosphate (Asc-2-P) (Wako, Richmond, Va.) or 50.0 μg/ml ascorbic acid (Sigma-Aldrich) and were added to the medium with the first and all subsequent medium changes. Alkaline phosphatase (ALP) activity and mineralized nodules were quantified as previously described (Gazit et al., J. Cell Biochem. 1998, 70:478-488; Gazit et al., J. Cell. Biochem. 1999, 73:379-389). Supplements for osteoclast differentiation included 30.0 nM soluble recombinant human receptor activator of nuclear factor-κB ligand (srhRANKL) (Research Diagnostics Inc., Flanders, N.J.) and 10.0 nM recombinant murine monocyte-colony stimulating factor (rmM-CSF) (R&D Systems, Minneapolis, Minn.) and were added to the medium with the first and all subsequent medium changes. For a review of the differentiation technique, see Bodine and Komm, Vitamins and Hormones 2002, 64:101-151.

Preosteoclasts (mononuclear cells) and mature osteoclasts (multinuclear cells, >2 nuclei/cell) were stained with tartrate-resistant acid phosphatase (TRAP) and quantified with a commercially available kit (Sigma-Aldrich) as previously described (Murrills et al., Methods in Bone Biology, T. R. Arnett, and B. Henderson, eds. (London, Chapman & Hall), 1998, 64-105). Staining for β-galactosidase was performed as follows. The cell layers were fixed at 4° C. for 10 min in phosphate buffered saline, pH 8.8, that contained 2.0% paraformaldehyde, 0.2% glutaraldehyde, 5.0 mM EGTA, pH 7.3, and 2.0 mM MgCl₂ as described by (Kim et al., Mechanisms of Development 1999, 80:159-170). β-galactosidase staining of the cell layers was determined after 4 hours of incubation at 37° C. using phosphate buffered saline, pH 7.2, that contained 5.0 mM potassium ferricyanide, 2.0 mM MgCl₂, 0.2% NP-40 (USB, Cleveland, Ohio), 0.01% sodium deoxycholate and 1.0 mg/ml X-Gal (Gold Biosciences, St. Louis, Mo.).

### Example 19

### Deletion of sFRP-1 Does Not Alter Bone Resorption or Skeletal Development

In order to determine if bone resorption was altered in vivo in the sFRP-1 −/− mice, 19-week old female −/− and +/+ animals were ovariectomized (in order to induce estrogen deficiency), necropsied 21 weeks later, and pQCT analysis of their tibia was performed. When compared to the sham operated −/− mice, the Ovx −/− mice exhibit decreased volumetric trabecular bone mineral density (a 39% decrease) as determined by this method. This indicates that bone resorption is normal in the −/− mice. Ovariectomy of the +/+ mice did not reduce trabecular vBMD, since by 40 weeks of age, this parameter is already low.

Although deletion of sFRP-1 in mice leads to increases in trabecular bone, it does not alter skeletal development. Faxitron radiographs (Faxitron X-Ray Corp., Buffalo Grove, Ill.) demonstrate that the skeletons of the 39-40-week old −/− mice look essentially the same as the +/+animals.

### Example 20

### Deletion of sFRP-1 Enhances Bone Marrow-Derived Osteoprogenitor Cell and Calvarial-Derived Osteoblast Proliferation

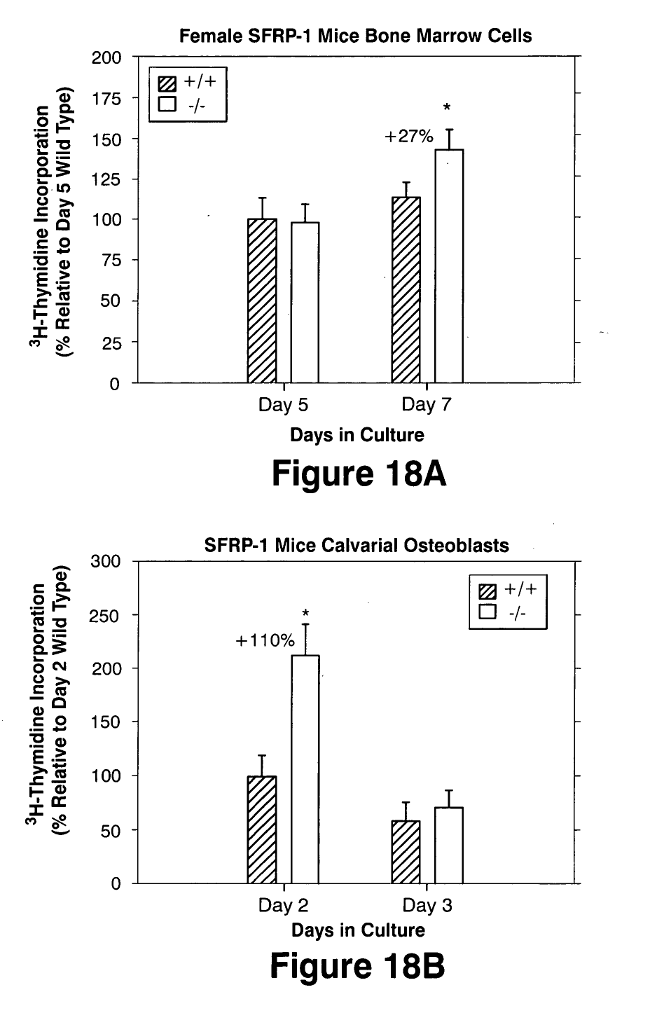

In order to determine if cellular proliferation was altered by deletion of sFRP-1, we isolated bone marrow from +/+ and −/− mice and performed an in vitro DNA synthesis assay, which is a surrogate marker for cell division. After 7 days in culture, cells from sFRP-1−/− mice exhibited a 27% increase in [³H]-thymidine incorporation when compared to cells from +/+controls (FIG. 18A). To confirm these results, DNA synthesis was also measured in neonatal calvarial-derived osteoblast cultures (FIG. 18B). During the proliferative phase (day 2), osteoblasts from sFRP-1−/− mice exhibited a 110% increase in [³H]-thymidine incorporation when compared to +/+ cells. However, the increased proliferation of the −/− cells returned to normal levels by day 3 when the cultures entered the differentiation phase. Thus, these results demonstrate that deletion of sFRP-1 in vivo leads to an enhancement of bone marrow-derived osteoprogenitor cell and calvarial-derived osteoblast proliferation in vivo.

### References Cited

Numerous references, including patents, patent applications and various publications, are cited and discussed in the description of this invention. The citation and/or discussion of such references is provided merely to clarify the description of this invention and is not an admission that any such reference is “prior art” to the invention described herein. All references cited and discussed in this specification are incorporated herein by reference in their entirety and to the same extent as if each reference was individually incorporated by reference.

What is claimed is:

1. A pharmaceutical composition for regulating bone-forming activity in a mammal comprising at least one of (i) a secreted frizzled related protein (sFRP) or regulating portion thereof (ii) an antibody against such proteins or portions thereof, (iii) a nucleic acid that encodes for either (i) or (ii); (iv) an sFRP antisense nucleic acid; or (v) a small molecule that has an effect on any of items (i)-(iv).

2. A pharmaceutical composition according to claim 1, wherein the sFRP is from human osteoblast cells.

3. A pharmaceutical composition according to claim 1, wherein the bone forming activity is the regulation of bone growth.

4. A pharmaceutical composition according to claim 1, wherein the bone forming activity is regulation of bone density.

5. The pharmaceutical composition according to claim 1, wherein the sFRP is sFRP-1.

6. The pharmaceutical composition of claim 1 wherein the composition comprises an acceptable carrier or diluent.

7. A method for treating a bone disorder in a mammal comprising the steps of administering a pharmaceutical composition as in claim 1.

8. The method of treating the bone disorder of claim 7, wherein the disorder comprises the group consisting of (a) a bone formation disorder, (b) a bone resorption disorder, and (c) a bone density disorder.

9. The method of claim 7 wherein the bone disorder is a degenerative bone disorder.

10. The method of claim 9 wherein the degenerative bone disorder is an osteodegeneration disorder.

11. The method of claim 10, wherein the osteodegeneration disorder is selected from the group consisting of osteopenia, osteoarthritis, osteoporosis.

12. The method of claim 7, wherein the mammal is a human.

13. A method for identifying a test compound that regulates sFRP activity, which method comprises determining activity of sFRP incubated in a medium containing a test compound, wherein an increase in activity relative to sFRP alone indicates the compound is an sFRP activator and a decrease in activity indicates the compound is an sFRP inhibitor.

14. The method of claim 13 wherein the sample comprises an immortalized human osteoblast cell that expresses a temperature-sensitive mutant of simian virus 40 large T protein antigen, wherein the cell proliferates at about 34° C. but does not proliferate at temperatures exceeding about 37° C., when the T-antigen mutant is inactive.

15. The method of claim 14 wherein the immortalized human osteoblast cell is an hOB-01-C1-PS-09 cell, as deposited with American Type Culture Collection in Manassas, Va. with the designation PTA-785, or progeny thereof.

16. A method of modulating Wnt-mediated signaling in a cell comprising contacting the cell with the composition of claim 1, wherein the Wnt activity is regulated.

17. The method of claim 16, wherein the sFRP of the composition is sFRP-1.

18. A method of facilitating bone formation or repair in a bone cell, comprising introducing a recombinant construct expressing an antisense, siRNA, shRNA sequence to a nucleotide sequence that encodes an sFRP-1 into bone cells.

19. A method of diagnosing a bone disease or disorder, the method comprising using a polynucleotide probe capable of hybridizing with the polynucleotide having the nucleic acid sequence set forth in SEQ ID NO: 1 to detect the presence or absence of an sFRP in a sample derived from a mammalian host.

20. A pharmaceutical composition for regulating bone-forming activity in a mammal comprising at least one antibody to a secreted frizzled related protein (sFRP) or regulating portion thereof.

21. The pharmaceutical composition of claim 20 wherein the composition comprises an acceptable carrier or diluent.

22. The pharmaceutical composition of claim 20 wherein the antibody is raised against at least 8 consecutive amino acids of an sFRP protein.

23. The pharmaceutical composition of claim 20 wherein the antibody is raised against at least 10 consecutive amino acids of an sFRP protein.

24. The pharmaceutical composition of claim 20 wherein the antibody is raised against at least amino acids 217-231 of an sFRP protein of SEQ ID NO: 2.

25. The pharmaceutical composition as in claim 1, wherein the sFRP has the amino acid sequence obtained by the expression of the polynucleotide sequence set forth in SEQ ID NO: 1.

26. A method for identifying a test compound that modulates sFRP activity, which method comprises comparing the phenotypic changes induced by the test compound on a sFRP +/+ animal with the phenotypic changes induced by the test compound on a sFRP −/− animal, wherein a phenotypic change in the sFRP +/+animal relative to the sFRP −/− animal indicates the compound is a modulator of sFRP activity.

27. An immortalized human osteoblast (hOB) cell that expresses a temperature-sensitive mutant of simian virus 40 large T protein antigen, wherein the cell proliferates at about 34° C. but does not proliferate at temperatures exceeding about 37° C., when the T-antigen mutant is inactive.

28. An hOB cell of claim 27 that expresses a nucleotide sequence encoding a polynucleotide that encodes an sFRP or fragment thereof.

29. An hOB cell of claim 27 wherein the hOB is an hOB-01-C1-PS-09 cell, as deposited with American Type Culture Collection in Manassas, Va. with the designation PTA-785, or progeny thereof.

30. A homogenous population of cells comprising the hOB cell of claim 27.

31. A method for preventing a bone disorder in a mammal, which method comprises administering a pharmaceutical composition as in claim 1.

32. The method of preventing a bone disorder according to claim 31, in which the disorder is a bone formation disorder, a bone resorption disorder or a bone density disorder.

33. The method according to claim 31 in which the disorder is a degenerative bone disorder.

34. The method according to claim 33 in which the degenerative bone disorder is an osteodegeneration disorders.

35. The method according to claim 34 in which the osteodegeneration disorder selected from the group consisting of osteopenia, osteoarthritis, and osteoporosis.

36. The method according to claim 35 in which the disorder is Type II osteoporosis.

37. A method according to claim 31 in which the mammal is a human.

38. A method according to claim 31 in which the pharmaceutical composition inhibits expression or activity of the sFRP in the mammal.

39. A method according to claim 38 in which the sFRP expression or activity is inhibited by at least 20%.

40. A method according to claim 38 in which the sFRP expression or activity is completely eliminated in the mammal.

41. A method according to claim 7 in which the pharmaceutical composition inhibits expression or activity of the sFRP in the mammal.

42. A method according to claim 41 in which the sFRP expression or activity is inhibited by at least 20%.

43. A method according to claim 41 in which the sFRP expression or activity is completely eliminated in the mammal.


SEQUENCE LISTING (30 sequences)

SEQ ID NO: 1 - DNA, 2602 bp, Homo sapiens
gatctgctggggactgcgccttttgtccccggaggtccctggaagtttgcggcgggacgcgcgcggggaggcggcggaggcagccccgacgtcgcggagaacagggcgcagagccggcatgggcatcgggcgcagcgaggggggccgccgcggggcagccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtgattctcccgggggcagggaattctgcagatatccagcatggggagggagcctcgggtggggtgggagcgggggggacagtgccccgggaacccggtgggtcacacacacgcactgcgcctgtcagtagtggacattgtaatccagtcggcttgttcttgcagcattcccgctcccttccctccatagccacgctccaaaccccagggtagccgtggccgggtaaagcaagggccatttagattaggaaggtttttaagatccgcaatgtggagcagcagccactgcacaggaggaggtgacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtagtagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcggtagttttcaaaacacatagttaaaaaagaaacaaatgaaaaaaattttagaacagtccagcaaattgctagtcagggtgaattgtgaaattgggtgaagagcttacgattctaatctcatgttttttccttttcacatttttaaaagaacaatgacaaacacccacttatttttcaaggttttaaaacagtctacattgagcatttgaaaggtgtgctagaacaaggtctcctgatccgtccgaggctgcttcccagaggagcagctctccccaggcatttgccaagggaggcggatttccctggtagtgtagctgtgtggctttccttcctgaagagtccgtggttgccctaaaacctaacaccccctagcaaaactcacagagctttccgtttttttctttcctgtaaagaaacatttcctttgaacttgattgcctatggatcaaagaaattcagaacagcctgcctgtccccccgcactttttacatatatttgtttcatttctgcagatggaaagttgacatgggtggggtgtccccatccagcgagagagtttcaaaagcaaaacatctctgcagtttttcccaagtgccctgagatacttcccaaagcccttatgtttaatcagcgatgtatataagccagttcacttagacaactttacccttcttgtccaatgtacaggaagtagttctaaaaaaaaa

SEQ ID NO: 2 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 3 - DNA, 16 bp, Artificial Sequence
  PCR primer
aagcttttttttttta

SEQ ID NO: 4 - DNA, 13 bp, Artificial Sequence
  PCR primer
aagcttgattgcc

SEQ ID NO: 5 - DNA, 21 bp, Artificial Sequence
  PCR primer
gctggggactgcgccttttgt

SEQ ID NO: 6 - DNA, 23 bp, Artificial Sequence
  PCR primer
cctgcccccgggagaatcactta

SEQ ID NO: 7 - protein, 313 aa, Homo sapiens
  ref <308>: DATABASE ACCESSION NUMBER: GenBank / NP_003003
  ref <309>: DATABASE ENTRY DATE: 2003-09-06
  ref <313>: RELEVANT RESIDUES: (1)..(313)
MGIGRSEGGRRGALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 8 - DNA, 18 bp, Artificial Sequence
  oligonucleotide
ggcatgggcatcgggcgc

SEQ ID NO: 9 - DNA, 18 bp, Artificial Sequence
  oligonucleotide
gcgcccgatgcccatgcc

SEQ ID NO: 10 - DNA, 19 bp, Artificial Sequence
  PCR primer
ctgaggctgtgccacaacg

SEQ ID NO: 11 - DNA, 19 bp, Artificial Sequence
  PCR primer
catgaccggctcgcacgag

SEQ ID NO: 12 - DNA, 30 bp, Artificial Sequence
  PCR primer
gcagcgggacgcgcgcgtgaaggcagcgtg

SEQ ID NO: 13 - DNA, 30 bp, Artificial Sequence
  PCR primer
ggggttcgcgggcgtgggaaggcataccct

SEQ ID NO: 14 - DNA, 22 bp, Artificial Sequence
  PCR primer
atgtgtatcttgagttggtatc

SEQ ID NO: 15 - DNA, 22 bp, Artificial Sequence
  PCR primer
cataatacttgcaaattgatgc

SEQ ID NO: 16 - DNA, 21 bp, Artificial Sequence
  PCR primer
caacatagcactacatcttcg

SEQ ID NO: 17 - DNA, 18 bp, Artificial Sequence
  PCR primer
ggccaacgctgaagccag

SEQ ID NO: 18 - DNA, 25 bp, Artificial Sequence
  PCR primer
ggcagccccgacgtcgccgagcaac

SEQ ID NO: 19 - DNA, 24 bp, Artificial Sequence
  PCR primer
ccttggggttagaggcttccgtgg

SEQ ID NO: 20 - DNA, 26 bp, Artificial Sequence
  PCR primer
acggcatggtgccaatgaatcgtctg

SEQ ID NO: 21 - DNA, 26 bp, Artificial Sequence
  PCR primer
caaataatatcggtggccgtggtgtc

SEQ ID NO: 22 - DNA, 19 bp, Artificial Sequence
  PCR primer
gccacaacgtgggctacaa

SEQ ID NO: 23 - DNA, 20 bp, Artificial Sequence
  PCR primer
acctctgccatggtctcgtg

SEQ ID NO: 24 - DNA, 23 bp, Artificial Sequence
  probe
agatggtgctgcccaacctgctg

SEQ ID NO: 25 - DNA, 24 bp, Artificial Sequence
  PCR primer
ctgctgatgaagcagaacaacttt

SEQ ID NO: 26 - DNA, 19 bp, Artificial Sequence
  PCR primer
gcgtgtaccacagcggatg

SEQ ID NO: 27 - DNA, 21 bp, Artificial Sequence
  probe
cgccgtgcgctgttcgcatta

SEQ ID NO: 28 - DNA, 20 bp, Artificial Sequence
  PCR primer
cgcttgtgctgttcctgaag

SEQ ID NO: 29 - DNA, 20 bp, Artificial Sequence
  PCR primer
cggcccatgatgagaaagtt

SEQ ID NO: 30 - DNA, 21 bp, Artificial Sequence
  probe
tgccgactgtccctgccacca